(19) United States
(12) Patent Application Publication
(10) Pub. No.: US 2026/0369410 A1
(43) Pub. Date: Nov. 19, 2026

(54) SYSTEM AND METHOD FOR AI BASED AUTOMATIC MEDICAL DATA EXTRACTION

(71) Applicant: IGENTIFY LTD., Caesarea (IL)

(72) Inventors: Ofir AMZALEG, Yokneam Illit (IL); Doron BEN-MOSHE, Gaaton (IL); Udi ZWILINSKY, Beit Aryeh (IL); Yair BRIMAN, Zichron Yaacov (IL); Doron BEHAR, Kerem Maharal (IL)

(73) Assignee: IGENTIFY LTD., Caesarea (IL)

(21) Appl. No.: 19/675,558

(22) Filed: May 13, 2026

Related U.S. Application Data
(60) Provisional application No. 63/804,730, filed on May 13, 2025.

(51) Int. Cl. G16H 50/20; G16H 15/00

(52) U.S. Cl. CPC G16H 50/20; G16H 15/00

(57) ABSTRACT

A system and method for extracting medical data from clinical reports includes: identifying a format of the clinical report; retrieving pipeline instructions associated with the report format, wherein the pipeline instructions includes a report template, at least one prompt and guard rules, and using the pipeline instruction: segmenting the clinical report into segments using the report template; for each segment, extracting data from the segment by applying the at least one extraction prompt to at least one LLM; evaluating the extracted data against guard rules; refining the extracted data based on the guard rule evaluation; receiving operator corrections made to previously extracted data; detecting, using the at least one LLM, patterns in the operator corrections; generating, using the at least one LLM, transformation rules that encode the patterns in the operator corrections into transformation rules; and applying the transformation rules to the extracted data to obtain normalized data.

### CROSS-REFERENCE TO RELATED APPLICATIONS

This application claims the benefit of U.S. Provisional Application Ser. No. 63/804,730, filed May 13, 2025, which is hereby incorporated by reference in its entirety.

### FIELD OF THE INVENTION

Embodiments of the present invention relate generally to the field of automatic data extraction using artificial intelligence (AI); for example extraction of medical data from clinical reports. Some embodiments relate to post processing techniques for refining the extracted data.

### BACKGROUND

In the U.S. healthcare system, lab results are often delivered via email, fax, electronic health records (EHR) integrated application programming interface (API), or lab portals. These methods suffer from several problems. Manually entering diverse report formats is slow and error-prone, increasing the risk of assigning incorrect results to the wrong patient. In addition, inconsistent workflows and lack of automation lead to delays and poor data quality.

Current attempts to automatically extract medical data from clinical reports typically provide inaccurate results that do not meet clinical standards. This may be attributed to the numerous report formats, and variability in medical terms usage. In addition, the format of a particular lab clinical report may change over time. Thus, a clinical data extraction system that was designed to extract data from the old format may fail with the new format.

Accordingly, a method that would accurately extract data from a variety of clinical reports, and that would be able to learn new formats and client preferences is required.

### SUMMARY

According to some embodiments, a computer-based system and method for extracting medical data from clinical reports may include using a processor: identifying a format of the clinical report using a trained classifier; retrieving pipeline instructions associated with the report format, wherein the pipeline instructions comprise a report template, at least one prompt and guard rules, and using the pipeline instruction: segmenting the clinical report into segments using the report template; for each segment, extracting data from the segment by applying the at least one extraction prompt to at least one large language model (LLM); evaluating the extracted data against guard rules; refining the extracted data based on the guard rule evaluation; receiving operator corrections made to previously extracted data; detecting, using the at least one LLM, patterns in the operator corrections; generating, using the at least one LLM, transformation rules that encode the patterns in the operator corrections into transformation rules; and applying the transformation rules to the extracted data to obtain normalized data.

According to embodiments, the guard rules may include: presence guards that verify that all data fields marked as mandatory in the pipeline instructions are populated with non-null values in the extracted data, range guard rules that verify that numeric data in the extracted data fall within clinically plausible ranges defined in the pipeline instructions, and format guards that verify that string data fields in the extracted data conform to expected patterns.

According to embodiments, types of the guard rules may include:
LLM-based guard rules that comprise an evaluation prompt comprising the extracted data and the segment from which the extracted data were extracted as context,
code-based guard rules that apply logic rules to the extracted data,
regex-based guard rules that validate format of extracted string data fields within the data by matching the extracted string data fields against one or more regular expression patterns,
rule-based guard rules that evaluate the extracted data against domain knowledge bases;
generic guard rules that validate date of birth (DOB), patient name and general results, and
clinical indication-specific guard rules that validate variant names and gene names.

According to embodiments, the transformation rules that target non-overlapping extracted data may be applied in parallel and the transformation rules that target the same extracted data may be applied sequentially.

According to embodiments, types of the transformation rules may include:
code transformation rules that encode transformation logic as executable software code,
regular expressions (regex) transformation rules that apply regular expression patterns to the data,
prompt transformation rules that comprise an LLM prompt that describes the transformation rule,
LLM-generated rules that are produced by an LLM rule synthesizer and encode transformation patterns inferred from operator correction behavior.

According to embodiments, refining the extracted data may include, in case of evaluating a first extracted data item against a first guard rule fails: providing to the at least one LLM the first extracted data item, the first guard rule, the segment including the first extracted data item and the extraction prompt that was applied to the at least one LLM to extract the first extracted data; updating, by the at least one LLM, the extraction prompt applied to the at least one LLM to extract the first extracted data item; and applying the updated extraction prompt to the segment including the extracted data item to obtain a refined extracted data item.

According to embodiments, refining the extracted data based on the evaluation, in case evaluating a first extracted data item against a first guard rule fails, may include any one of:
for cases of semantic mismatch and missing value failure modes: updating the extraction prompt used to extract the extracted data item to comprise a guard failure reason, constraining the extracted data to the expected output format, and applying the updated extraction prompt to the segment including the first extracted data item to extract a refined first data item from the updated segment,
for missing-value failures: repeating segmenting the clinical report into segments with adjusted boundary parameters, and, for the updated segment including the first extracted data item, repeating applying the extraction prompt to the at least one LLM to extract a refined first data item from the updated segment;
for data that follow deterministic patterns-applying regex rules to the segment including the first extracted data item to extract a refined first data item from the updated segment,
for complex segments that contain multiple overlapping data items: segmenting the segment including the first extracted data item into smaller, non-overlapping sub-segments, applying independent extraction prompts to each sub-segment, and merging the resulting data to extract a refined first data item from the updated segment.

Embodiments may include, evaluating the normalized data against a second set of guard rules; and refining the normalized data based on the evaluation of the normalized data against a second set of guard rules.

According to embodiments, refining the normalized data based on the evaluation of the normalized data against the second set of guard rules may include, in case evaluating a first normalized data item against a second guard rule fails: providing the first normalized data item, the second guard rule, the extracted data item before normalization, and the transformation rule that was applied to the extracted data item to perform the normalization to the at least one LLM;
updating, by the at least one LLM, the transformation rule that was applied to the extracted data item to perform the normalization; and applying the updated transformation rule to the extracted data item before normalization to obtain a refined normalized data item.

Embodiments may include, detecting whether there are deviations from the report template within the clinical report using anomaly detection rules, before segmenting the clinical report; and providing an alert in case deviations are detected.

According to embodiments, in a preparation stage: receiving a set of labelled clinical reports of the clinical report format, wherein the label comprising true segment boundaries and true data values; and generating, using at least one machine learning model, the pipeline instructions for the clinical report format.

According to embodiments, generating the pipeline instructions for the clinical report format may include: training a machine learning classifier using the set of labelled clinical reports;
identifying segments in the clinical report format and extracting segment boundaries for each of the segments; generating anomaly detection rules for the clinical report format; generating, from the set of labelled clinical reports using a large language model (LLM), the at least one extraction prompt for each of the segments, wherein each of the extraction prompts is configured to extract data from a segment of the clinical report format; analysing the distribution of the true data values to generate guard rules that enable define value ranges for extracted data; and generating an initial set of transformation rules based on data patterns in the set of labelled clinical reports.

### BRIEF DESCRIPTION OF THE DRAWINGS

For a better understanding of embodiments of the disclosure and to show how the same can be carried into effect, reference will now be made, purely by way of example, to the accompanying drawings in which like numerals designate corresponding elements or sections throughout.

In the accompanying drawings:

FIG. 1 is a block diagram of an exemplary computing device which may be used with some embodiments.

FIG. 2 is a block diagram of a system for extracting medical data from clinical reports, according to some embodiments.

FIG. 3A provides an example of a segmented clinical report, according to some embodiments.

FIG. 3B provides an example of extracted data, according to some embodiments.

FIG. 4 is a flowchart of a method for extracting medical data from clinical reports, according to embodiments.

FIG. 5 is a flowchart of a method for performing onboarding for a new clinical report format, according to some embodiments.

FIG. 6 is a block diagram of a computerized medical system, according to some embodiments.

It will be appreciated that, for simplicity and clarity of illustration, elements shown in the figures have not necessarily been drawn to scale. For example, the dimensions of some of the elements may be exaggerated relative to other elements for clarity. Further, where considered appropriate, reference numerals may be repeated among the figures to indicate corresponding or analogous elements.

### DETAILED DESCRIPTION

In the following detailed description, numerous specific details are set forth in order to provide a thorough understanding of the disclosure. However, it will be understood by those skilled in the art that the present disclosure can be practiced without these specific details. In other instances, well-known methods, procedures, and components, modules, units and/or circuits have not been described in detail so as not to obscure the disclosure.

According to embodiments of the invention, artificial intelligence (AI) or machine learning (ML) models disclosed herein, also referred herein as networks, may include one or more artificial neural networks (NN). NNs are models of systems made up of computing units typically called neurons (which are artificial neurons or nodes, as opposed to biological neurons) communicating with each other via connections, links or edges. In common NN implementations, the signal at the link between artificial neurons or nodes can be for example a real number, and the output of each neuron or node can be computed by function of the (typically weighted) sum of its inputs, such as a rectified linear unit (ReLU) function. NN links or edges typically have a weight that adjusts as learning or training proceeds typically using a loss or cost function, which may for example be a function describing the difference between a NN output and the ground truth (e.g., correct answer). The weight may increase or decrease the strength of the signal at a connection. Typically, NN neurons or nodes are divided or arranged into layers, where different layers can perform different kinds of transformations on their inputs and can have different patterns of connections with other layers. NN systems can learn to perform tasks by considering example input data, generally without being programmed with any task-specific rules, being presented with the correct output for the data, and self-correcting, or learning using the loss function. A NN may be configured or trained for a specific task, e.g., interpret and generate language. Training a NN for the specific task may involve adjusting these weights based on examples (e.g., labeled data included in the training dataset). Each neuron of an intermediate or last layer may receive an input signal, e.g., a weighted sum of output signals from other neurons, and may process the input signal using a linear and/or nonlinear function (e.g., an activation function). The results of the input and intermediate layers may be transferred to other neurons and the results of the output layer may be provided as the output of the NN. For example, in a NN algorithm known as the gradient descent algorithm, the results of the output layer may be compared to the labels of the samples in the training dataset, and a loss or cost function (such as the root-mean-square error) may be used to calculate a difference between the results of the output layer and the labels. The weights of some of the neurons may be adjusted using the calculated differences, in a process that iteratively minimizes the loss or cost until satisfactory metrics are achieved or satisfied.

A processor, e.g., central processing units (CPU), graphical processing units or fractional graphical processing units (GPU), tensor processing units (TPU) or a dedicated hardware device may perform the relevant calculations on the mathematical constructs representing the NN. As used herein an NN may include deep neural networks (DNN), convolutional neural networks (CNN), recurrent neural networks (RNN), long short-term memory networks (LSTM) probabilistic neural networks (PNN), time delay neural network (TDNN), deep stacking network (DSN), generative adversarial networks (GAN), Siamese networks, etc. Some embodiments of the invention may include other deep architectures such as transformers, that may include series of layers of self-attention mechanisms and feedforward neural networks, used for processing input data. Transformers may be used in light of their capacity of parallelism and their multi-headed self-attention which facilitate features extraction.

A large language model (LLM) may include a computational ML model capable of analyzing and generating general-purpose language and other natural language processing tasks. LLMs may learn, through a training process, statistical relationships between language elements from vast amounts of text. LLMs may include artificial NNs that utilize the transformer architecture. A transformer is a type of NN architecture capable of learning context and relationships from sequential data such as text. A transformer may include a concatenation of many transformer blocks, each composed of an attention component followed by a NN. LLMs may be trained on massive corpora of text to learn the statistical patterns of language. They are designed to model the probability distribution of token sequences, enabling them to generate, complete, and understand text.

An embedding model, found at the core of an LLM's functionality, is a model configured or trained to convert raw text into a numerical form called embeddings, which capture semantic and syntactic information about words, phrases, or longer pieces of text. Embeddings are dense vector representations of discrete linguistic units (e.g., tokens, words, sentences, or documents). Embeddings map symbolic input (e.g., the word “cat”) into a high-dimensional vector space where embeddings of similar meanings lie closer together. Embeddings may be used to predict the next token in a sequence or fill in masked tokens and may serve as the model's internal understanding of meaning. An example for an embedding model may include OpenAI text-embedding-3-large. Other embedding models may be used.

A completion LLM is a type of an LLM used in natural language processing (NLP) to generate text or complete text based on a given input prompt. It predicts the most likely continuation of a piece of text by using statistical patterns learned during training. Essentially, a completion model takes a piece of text and tries to predict what comes next, producing a likely continuation based on its training data. The completion model is characterized by an input-output behavior, where the input to the completion model may include a text prompt (e.g., a question, sentence fragment, or even just a few words), and the output may include a continuation of the text based on the model's understanding and training. Examples of completion models include OpenAI's original GPT models (like GPT-2 and GPT-3) in their raw API form and OpenAI gpt4-turbo. Completion mode in OpenAI's API (e.g., using text-davinci-003) involves feeding or inputting a prompt and getting a generated completion.

Reasoning LLMs or reasoning models are a type of an LLM that are specifically designed to perform complex reasoning tasks. Unlike completion model LLMs, which primarily generate text based on statistical patterns, reasoning LLMs attempt to break down problems into smaller steps, employ chains of thought, and sometimes even verify their own solutions before providing an answer. This makes them more human-like in their approach to problem-solving and enables them to tackle more complex challenges. Reasoning LLMs may simulate reasoning, drawing conclusions, solving problems, making decisions, or performing multi-step thinking based on patterns the model has learned from large-scale text data. While traditional reasoning models often rely on formal logic or explicit rules, reasoning LLMs may perform reasoning implicitly through learned representations, often guided by prompting or structured input. Reasoning LLMs may perform reasoning through text generation, e.g., predicting the next word or token based on a prompt. However, through this mechanism, the reasoning LLMs can simulate various modes of reasoning. An example for a reasoning LLM or a reasoning model may include OpenAI o1. Other reasoning models may be used. As used herein, reasoning models refer to reasoning LLMs.

Embodiments may improve existing LLM technology to automatically extract data from “noisy” input, for example extracting medical data from clinical reports. The clinical reports may include text describing medical information such as medical tests results and analysis, description of the performed tests, data identifying the patient, data identifying the laboratory or other medical facility that has performed the medical test, and other relevant information. Clinical reports may be provided in any applicable format such as PDF, DODX Word documents, electronic publication (EPUB) formats, HyperText markup language (HTML) pages, image-based formats, flipbook formats, and any other applicable format. Embodiments may perform a series of steps that would ensure accurate data extraction from a variety of clinical reports types and formats. The extracted data may be provided to other systems and databases in standard format such as a structured Json (JavaScript Object Notation) output or database record. Thus, clinical reports of a verity of clinical report formats may all be converted into a single standard form, enabling unified analysis and record keeping for healthcare organizations. In a preparation stage, pipeline instructions (also referred to as extraction configuration) that are specific to a certain type or format of clinical report may be generated. The pipeline instructions may include a report template, one or more prompts, one or more guard rules, one or more transformation rules, and other metadata that may be required for extracting data from the specific report format.

Embodiments may include an automatic new report format onboarding process that enables the system to autonomously generate the pipeline instructions for a previously unseen report format. The process receives a labelled and classified corpus of representative clinical reports of the target format, each annotated with ground-truth segment boundaries, extracted data, and extraction notes. An autonomous LLM-based agent may orchestrate a sequential pipeline of learning stages, iterating through each stage until its acceptance criterion is met before progressing to the next. Successful completion of all stages provides a full set of pipeline instructions that may be used to extract data form clinical reports having the new report format during routine operation.

During routine operation, when a clinical report is being analysed, an embodiment may first identify the type or format of the clinical report. Once the type or format of the clinical report is identified, pipeline instructions that are tailored specifically to the report type or format are retrieved. The report template included in the pipeline instructions may be used to segment or divide the clinical report into segments. The one or more prompts included in the pipeline instructions may be used to extract data from each segment by applying the one or more prompts to one or more LLMs.

Following the data extraction stage, a series of tests, evaluations and transformations are performed to verify the accuracy of the extracted data and refine the extracted data where needed. For example, the extracted data may be evaluated against guard rules, and the extracted data may be refined based on the evaluation results. Additionally or alternatively, post-processing transformation rules may be applied on the extracted data.

Embodiments of the invention may include automatic learning capabilities that may improve AI technology and further improve the accuracy of the extracted data. Some embodiments may provide or present the extracted results to a human operator. In case the human operator corrects the extracted data, embodiments may receive those operator corrections, detect patterns in the operator corrections using one or more LLMs, and generate, using the one or more LLMs, post-processing transformation rules that encode the patterns in the operator corrections into transformation rules. Those self-learned transformation rules may be applied to the extracted data to obtain normalized data. Additionally or alternatively, patterns may be detected in the operator corrections and one or more refined extraction prompts or transformation rules may be generated based on the detected patterns, using one or more LLMs, where the refined extraction prompts or transformation rules may encode the patterns in the operator corrections. The refined extraction prompts may be applied to the relevant segment to re-extract the data. Accordingly, embodiments may improve the technology of LLM prompt engineering by learning patterns in the user corrections and automatically transforming those patterns into LLM prompts (either extraction prompts or transformation rule prompts) that may in-turn improve the accuracy of the extracted data compared to current extraction systems. Current extraction systems do not learn from used corrections and do not employ prompt engineering based on user corrections and therefore may provide extracted data that does not fit the user needs and preferences.

The self-learning capabilities and the automatic extraction prompt generation may improve the technology of data extraction (e.g. medical data from clinical reports), by automatically adjusting to new or changing clinical report formats. While other data extraction systems may fail or provide poor results wherever the format of a clinical report even slightly changes or may be totally incapable of analyzing new types of reports, embodiments may employ AI and LLM technology to automatically adjust to changing or new formats.

While described with relation to extracting data from clinical reports, embodiments can be extended beyond medical use cases. While an example implementation focuses on medical document extraction and clinical interpretation, an embodiment of a core architecture, combining document parsing, structured data extraction, validation, and decision-support logic, can be applied to multiple domains. Examples may include:
Insurance: embodiments may be used for extracting claims data and automatically trigger approvals, rejections, or fraud investigations.
Finance: embodiments may be used for processing financial documents (e.g., bank statements, tax forms) to generate risk scores and initiate compliance actions
Legal: embodiments may be used for extracting contract terms and triggering alerts, approvals, or workflow actions (e.g., renewals, escalations).
Supply Chain: embodiments may be used for parsing invoices and shipment documents to update systems, release payments, or flag discrepancies.

In all these and other possible use cases, embodiments not only extract and structure data but also drive downstream decision-making and automation.

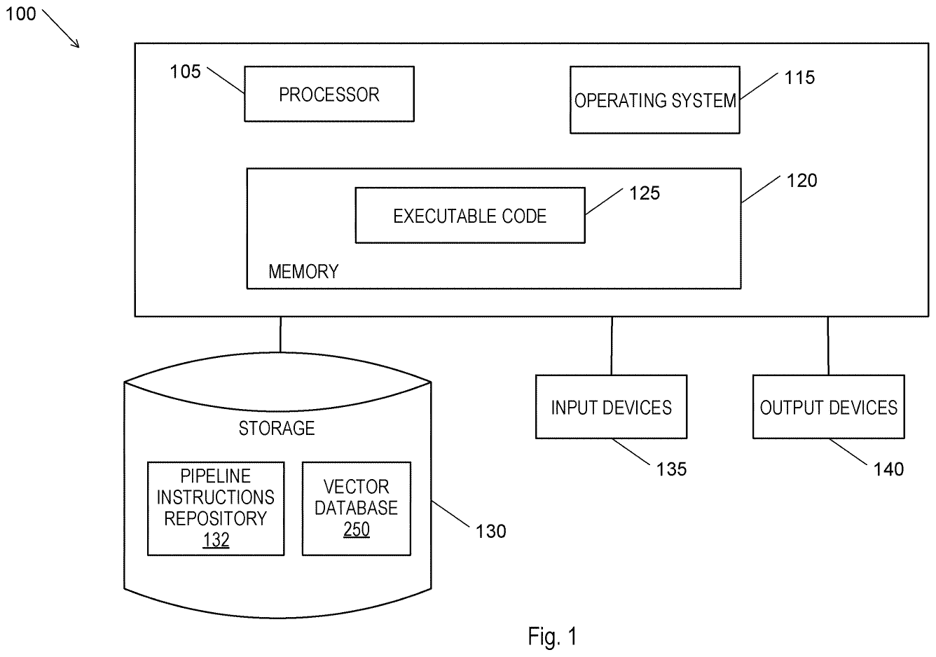

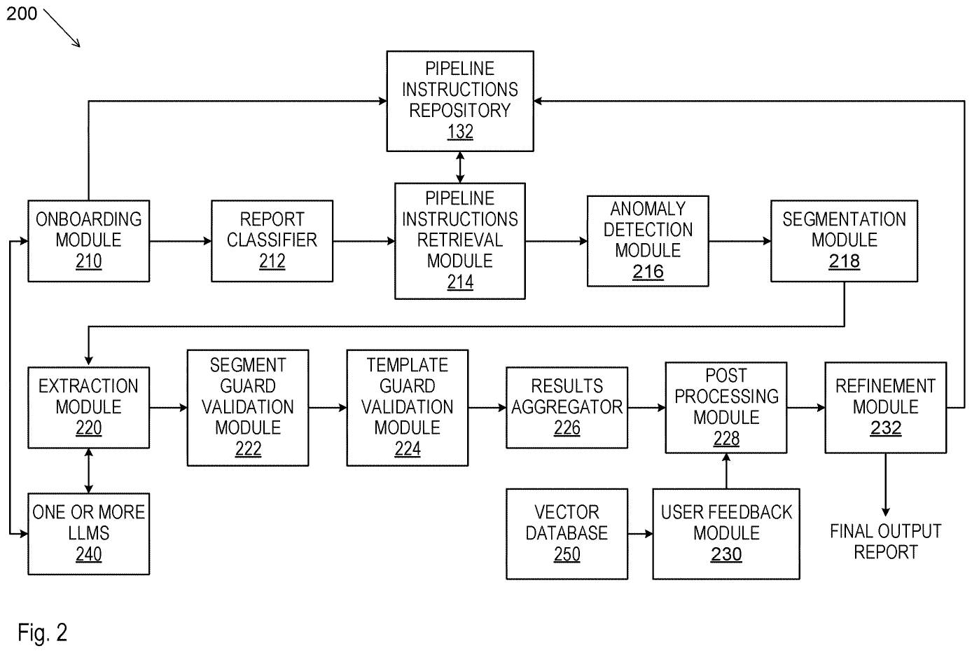

Reference is now made to FIG. 1, which is a block diagram of an exemplary computing device which may be used with some embodiments. Computing device 100 may include a controller or processor 105 that may be or include, for example, logic, one or more central processing unit processor(s) (CPU), one or more graphics processing unit(s) (GPU), one or more data processing unit(s) (DPU), one or more application-specific integrated circuits (ASICs), one or more field programmable arrays (FPGAs), one or more integrated circuits (ICs), a system-on-chip (SoC), a chip or any suitable computing or computational device, an operating system 115, a memory 120, a storage 130, input devices 135 and output devices 140, all connected to each other. Each of modules and equipment such as onboarding service 210, report classifier 212, pipeline instructions retrieval module 214, anomaly detection module 216, segmentation module 218, extraction module 220, segment guard validation module 222, template guard validation module 224, results aggregator 226, post processing module 228 and refinement module 232 shown in FIG. 2, and other modules or equipment mentioned herein may be or include, or may be executed by, a computing device such as included in FIG. 1 or specific components of FIG. 1, although various units among these entities may be combined into one computing device. In one embodiment, each of the above-listed modules comprise software that is used to program one or more processors (e.g., processor 105) to perform the functions described herein.

Operating system 115 may be or may include any code segment designed and/or configured to perform tasks involving coordination, scheduling, arbitration, supervising, controlling or otherwise managing operation of computing device 100, for example, scheduling execution of programs. Memory 120 may be or may include, for example, a random access memory (RAM), a read only memory (ROM), a dynamic RAM (DRAM), a synchronous DRAM (SD-RAM), a double data rate (DDR) memory chip, a Flash memory, a volatile memory, a non-volatile memory, a cache memory, a buffer, a short term memory unit, a long term memory unit, or other suitable memory units or storage units. Memory 120 may be or may include a plurality of possibly different memory units. Memory 120 may store for example, instructions to carry out a method (e.g., executable code 125), and/or data.

Executable code 125 may be any executable code, e.g., software or firmware, an application, a program, a process, task or script (e.g., any of the above-listed modules). Executable code 125 may be executed by processor 105 possibly under control of operating system 115. In some embodiments, more than one computing device 100 or components of device 100 may be used for multiple functions described herein. For the various modules and functions described herein, one or more computing devices 100 or components of computing device 100 may be used. Devices that include components similar or different to those included in computing device 100 may be used, and may be connected to a network and used as a system. One or more processor(s) 105 may be configured to carry out embodiments by, for example, executing software or code.

Storage 130 (which is non-transitory) may be or may include, for example, a hard disk drive, a floppy disk drive, a compact Disk (CD) drive, a CD-recordable (CD-R) drive, a universal serial bus (USB) device or other suitable removable and/or fixed storage unit. Data, e.g., pipeline instructions, may be stored in a storage 130 and may be loaded from storage 130 into a memory 120 where it may be processed by processor 105. In some embodiments, some of the components shown in FIG. 1 may be omitted. Storage 130 may store data required for performing embodiments of the invention, such as parameters and weights of LLMs. In addition, storage 130 may store databases and/or data repositories such as pipeline instructions repository 132 and vector database 250.

Input devices 135 may be or may include a mouse, a keyboard, a touch screen or pad or any suitable input device. It will be recognized that any suitable number of input devices may be operatively connected to computing device 100 as shown by block 135. Output devices 140 may include one or more displays, speakers and/or any other suitable output devices. It will be recognized that any suitable number of output devices may be operatively connected to computing device 100 as shown by block 140. Any applicable input/output (I/O) devices may be connected to computing device 100, for example, a wired or wireless network interface card, a modem, printer or facsimile machine, a universal serial bus (USB) device or external hard drive may be included in input devices 135 and/or output devices 140.

Embodiments may include one or more article(s) (e.g., memory 120 or storage 130) such as a computer or processor non-transitory readable medium, or a computer or processor non-transitory storage medium, such as for example a memory, a disk drive, or a USB flash memory, encoding, including or storing instructions, e.g., computer-executable instructions, which, when executed by a processor or controller, carry out methods disclosed herein.

Reference is made to FIG. 2, which is a block diagram of a system 200 for extracting medical data from clinical reports, according to some embodiments. It should be understood in advance that the components and functions shown in FIG. 2 are intended to be illustrative only and embodiments are not limited thereto. While in some embodiments the system of FIG. 2 is implemented using systems as shown in FIG. 1, in other embodiments other systems and equipment can be used.

One or more LLMs 240 may include any type of LLMs used by system 200, such as completion LLMs and reasoning LLMs, or any other types of LLMs.

Onboarding module 210 may automatically generate pipeline instructions, also referred to as extraction configuration, for a new (e.g., unseen by or unfamiliar to system 200) clinical report types or formats. Onboarding module 210 may automatically generate the pipeline instructions using a set of annotated or labelled clinical reports of the clinical report format, where the label may include ground-truth extraction results and/or metadata. The ground-truth extraction results or metadata may include an output schema and true data values and/or other ground truth extraction results or metadata. The output schema may define the structure of the clinical report, including true segment boundaries and data field names. The set of labelled clinical reports may be prepared offline, e.g., manually. The process of generating the pipeline instructions may include several stages of training ML models such as classifiers, generating prompts for LLM models and generating various types of rules. Each stage may be performed until the results provided by this stage meet an acceptance criterion. Upon successful completion of all stages, the full pipeline instructions may be associated with a template identifier and stored in pipeline instruction repository 132, also referred to as extraction configuration repository, and becomes available to other modules of system 200. This process may improve the technology of automatic data extraction by enabling extraction of data from new types of clinical reports and improving extraction accuracy and robustness.

Onboarding module 210 may train an ML classifier using the set of labelled clinical reports. Training the ML classifier using the set of labelled clinical reports may include performing a structural analysis of the set of labelled clinical reports to generate a template structure model, extracting candidate features from the template structure model, and training the ML classifier using the candidate features to determine whether an examined clinical report conforms to the clinical report format.

Onboarding module 210 may perform a structural analysis of the labelled set of clinical reports. The structural analysis may include clustering the clinical reports in the labelled set by visual layout similarity, font pattern distributions, and header and footer signatures to identify the dominant structural regions present across the set of labelled clinical reports. Recurring structural regions such as a report header, patient demographics block, results table, clinical summary, and appendix may be identified by frequency analysis of annotated segment boundaries. The structural analysis may produce a template structure model that may capture page layout topology, canonical section ordering, and report-level formatting rules. The template structure model may serve as the foundational reference for the subsequent stages.

Onboarding module 210 may identify discriminative features that may enable system 200 to classify incoming clinical reports as belonging to a particular clinical report format. The features identified in the structural analysis may be used to train an ML report classifier 212, that may be or may include a support vector machine (SVM), a random forest classifier, an ML or NLP algorithms trained on labeled clinical reports, or an LLM based classifier with few-shot prompting, executed by, for example, amazon web services (AWS) comprehend (an AWS NLP service). Onboarding module 210 may generate a classifier configuration, including feature weights, a decision boundary, and a calibrated confidence threshold below which a classification result is considered ambiguous and flagged for human review. During runtime, the classifier generated by onboarding module 210 may be used by report classifier 212.

Onboarding module 210 may identify segments or sections in the clinical report format and extract segment boundaries or divisions for each of the segments. The segments may include, for example, patient demographics, test results, clinical interpretations, etc. Extracting the per-segment boundaries may include analyzing the set of labelled clinical reports to extract textual and positional signals that delimit segment types within the clinical report format, generating a set of regular expression boundary rules and/or an LLM segmentation prompt that instructs an LLM of one or more LLMs 240, to identify segment start and end markers (e.g., segment titles), validating the set of regular expression boundary rules and the LLM segmentation prompt against a second set of labelled clinical reports having the clinical report format, and iteratively adjusting the set of regular expression boundary rules and the LLM segmentation prompt until a minimum coverage threshold is achieved. Onboarding module 210 may calculate segment importance weight reflecting the relative importance of each segment. Onboarding module 210 may generate a canonical segment name (e.g., results table, clinical summary, variants details, patient demographics), one or more heading keyword patterns used by a regular expressions (regex) detector, positional notes indicating the typical location of the segment within a clinical report of that template (e.g., top third, bottom half) from the extract textual and positional signals. Segmentation results may be included in the report format included in the pipeline instructions. For each expected segment, the report format may include the regular expression boundary rules, the LLM segmentation prompt, the segment list, the segment boundaries, the segment importance weight, the canonical segment name, the one or more heading keyword patterns used by the regex detector, the positional notes, and a minimum confidence threshold below which an LLM segment detection is not accepted. The report template may be included in the pipeline instructions.

Onboarding module 210 may generate the anomaly detection rules for the clinical report format by analyzing the distribution of data values and segment presence patterns in the set of labelled clinical reports. Generating the anomaly detection rules may include identifying which segments and data fields (e.g., placeholders for data items) are mandatory in the clinical report format, generating a presence checklist that defines which absences constitute anomalies, deriving plausible value ranges and canonical format patterns for data items in the clinical report format from the set of labelled clinical reports, and generating a structured anomaly detection configuration that includes missing-segment alert rules based on the presence checklist, out-of-range value flags based on the plausible value ranges, and format violation rules based on the canonical format patterns. The generated anomaly detection rules may be included in the pipeline instructions. During runtime, the generated anomaly detection rules may be used by anomaly detection module 216.

Onboarding module 210 may generate, from the set of labelled clinical reports using an LLM of one or more LLMs 240, an extraction prompt for each of the segments, wherein each of the extraction prompts is configured to extract data from a segment of the clinical report format. Prompts may be input to AI (e.g. LLMs) to produce output from the AI. The extraction prompts may include a data field schema and an expected output format. Generating each of the extraction prompts may include iteratively refining the extraction prompt by applying the extraction prompt to the associated segment to extract data from the segment, evaluating the extracted data against the true data values, and using an LLM of one or more LLMs 240 to refine the extraction prompt based on the evaluation. Onboarding module 210 may evaluate the generated extraction prompt against a second set of labelled clinical reports, compute accuracy scores such as data field-level F1 scores, and regenerate the extraction prompt with targeted corrections until all data field scores meet an acceptance threshold. This process may ensure that the generated LLM extraction prompts are designated to extract only the minimal, specific pieces of data present in the target segment. The extraction prompts may be included in the pipeline instructions. During runtime, the extraction prompts may be used by extraction module 220.

Onboarding module 210 may generate guard rules that define value ranges for extracted data. Generating the guard rules may include analyzing the distribution of the true data values and determining the guard rules based on the analysis. The generated guard rules may include an LLM guard prompts that instruct an LLM of one or more LLMs 240 to evaluate semantic plausibility, a code guard function that performs deterministic range and type checking, and a regular expression guard pattern that validates format compliance. The generated guard rules may be included in the pipeline instructions. During runtime, the generated guard rules may be used by segment guard validation module 222 and template guard validation module 224. Some guard rules may be generated manually and provided to onboarding module 210 in addition to the labelled clinical reports. In some embodiments onboarding module 210 may

Onboarding module 210 may generate an initial set of transformation rules (also referred to as post processing rules) based on data patterns observed by the module or by an AI system in the set of labelled clinical reports. The initial set of transformation rules may include transformation rules for formatting conventions, for unit normalization, for date format standardization, for vocabulary mappings, and for type coercion. Onboarding module 210 may generate the initial set of transformation rules for the clinical report format by inferring normalization transformations from the patterns observed in set of labeled clinical reports. Onboarding module 210 may identify systematic formatting conventions such as unit normalization requirements, date format standardization, controlled vocabulary mappings, and type coercion rules. Onboarding module 210 may translate those observations into transformation rules Additionally or alternatively, onboarding module 210 may configure triggers for post processing module 224, specifying the minimum correction frequency that must be observed for a data item before automatic rule synthesis is attempted. The transformation rules and/or the minimum correction frequency may be included in the pipeline instructions. During runtime, the transformation rules may be used by post processing module 228.

Onboarding module 210 may configure or create refinement loop parameters that may define how refinement module 232 behaves for this clinical report format. Onboarding module 210 may analyze the distribution of confidence scores observed across date extracted from the labeled set of clinical reports to set per-data field acceptance thresholds below which refinement module 232 is triggered. Onboarding module 210 may set maximum iteration budgets and escalation policies for refinement module 232, ensuring that refinement cycles are bounded and that unresolvable extraction failures are escalated to human review rather than silently producing low-confidence outputs. The refinement loop parameters may be included in the pipeline instructions.

Upon generating the complete pipeline instructions, onboarding module 210 may execute an end-to-end validation pass over a second set of labeled clinical reports (e.g., labeled clinical reports of the same clinical report format that were not used by onboarding module 210 to generate the pipeline instructions). The full extraction pipeline including segmentation, extraction prompts, guard rules, transformation rules and refinement policies, may be applied to the second set of labeled clinical reports, and the resulting data may be evaluated against the ground-truth extraction results. The validation may be considered successful only if a success criterion is met, e.g., if all data-level F1 scores meet their configured thresholds. If any data-level F1 score falls below threshold, onboarding module 210 may repeat one or more of the previously described operations, and revalidate until the validation is successful. Once the validation is successful, the complete pipeline instructions are stored in pipeline instructions repository 132.

Report classifier 212 may be an ML classifier or a plurality of ML classifiers trained to identify a type of a clinical report. In some embodiments, report classifier 212 may be generated in a preparation stage by onboarding module 210 and used during routine operation to classify incoming clinical reports, e.g., to identify a type or format of the clinical report. Classifier 212 may be an ML or NLP algorithm trained on labeled clinical reports, an LLM based classifier with few-shot prompting implemented, for example on AWS comprehend (an AWS NLP service), a support vector machine or a random forest classifier.

Upon receipt of an incoming clinical report, a pre-processing application may perform a feature extraction pass over three extraction zones of the clinical report: (i) the title zone, (ii) the page header, and (iii) the page footer. These extraction zones typically carry report-type specific textual signals, such as laboratory identifiers, diagnostic test names, department designations, accreditation codes, and standardized form headings, that collectively provide a high-fidelity fingerprint sufficient to distinguish among the known set of clinical report formats registered in system 200.

Each incoming clinical report may be parsed by the pre-processing application to isolate text from the various (e.g. three) extraction zones. The title zone typically captures the dominant heading text of the first page, typically the formal name of the report format (e.g., “Comprehensive Genomic Profiling Report,” “Pathology Report,” “Clinical Chemistry Results”). The header zone typically captures recurring top-of-page content across all pages of the clinical report, including laboratory name, institutional logo text, report identifier, and date fields. The footer zone typically captures page-level repeating content such as department codes, accreditation numbers, legal disclaimers, or version identifiers. Text extracted from each zone is independently normalized, e.g., lowercased, stripped of non-semantic punctuation, and whitespace-collapsed, before being concatenated into a single feature string.

The feature string may be converted into a numeric representation suitable for model inference. In the standard ML configuration, this is achieved using a term frequency-inverse document frequency (TF-IDF) vectorizer fitted over the labeled training corpus of known report templates. Each clinical report may be represented as a sparse vector encoding the relative importance of terms drawn from the title, header, and footer zones. Where semantic similarity across template variants is required, a dense embedding representation may be produced by a pre-trained sentence encoder, and concatenated with the TF-IDF sparse vector to form a hybrid feature representation.

The feature vector may be passed to classifier 212 trained on a set of labeled clinical reports (e.g., by onboarding module 210). Classifier 212 may output a probability distribution over all known clinical report formats. A confidence threshold may govern acceptance: if the highest-probability class meets or exceeds the threshold, the corresponding template identifier is selected as the resolved clinical report format. If no class exceeds the threshold, the clinical report may be routed to anomaly detection module 216 and may be flagged for human-in-the-loop review. In deployments where the set of labeled clinical reports is sparse or report format diversity is high, an LLM-based fallback classifier employing few-shot prompting may be invoked as a secondary resolver, providing generalization capability over previously unseen or newly introduced report formats.

Upon successful classification, classifier 212 may provide pipeline instructions retrieval module 214 a structured resolution record including the resolved template identifier, the associated confidence score, and the extracted title, header, and footer strings used in the classification decision. Pipeline instructions retrieval module 214 may use the template identifier to retrieve the corresponding pipeline instructions associated with identified report from pipeline instructions repository 132. The resolution record is also made available to anomaly detection module 216 to enable monitoring changes across clinical report batches. Pipeline instructions retrieval module 214 may provide the retrieved pipeline instructions to other modules in system 200.

Anomaly detection module 216 may detect whether there are deviations from the report template (included in the pipeline instructions) within the clinical report using anomaly detection rules, before segmenting the clinical report, and provide an alert in case deviations are detected.

Anomaly detection module 216 may ensure the integrity of the extraction process by detecting structural or formatting anomalies within incoming clinical reports. Anomaly detection module 216 may identify instances where a report format of the incoming clinical report has been altered in a manner that might lead to erroneous data extraction. Upon detecting such an anomaly, anomaly detection module 216 may trigger an alert to the human-in-the-loop interface, preventing the delivery of incorrect results and flagging the new format for prompt refinement or onboarding process.

Anomaly detection module 216 may process each incoming clinical report immediately after the incoming clinical report has been classified by report classifier 212. Anomaly detection module 216 may verify that the incoming clinical report conforms to the structural expectations of its report template before any data extraction is attempted. Anomaly detection module 216 may verify the structure of the incoming clinical report by checking for the presence of a set of pre-configured named segments that are known to exist in that report template, as described in the pipeline instructions. If one or more expected segments are absent or undetectable, Anomaly detection module 216 may conclude that the template of the incoming clinical report may have been modified, corrupted, or not yet registered, and raises an alert accordingly.

Each report template type registered in system 200 may be associated with a segment list included in the report template section in the pipeline instructions. The segment list may include the segments that must be present in a clinical report conforming to the report format, together with a per-segment importance weight reflecting the relative importance of that segment. For example, a report template of type “Oncology Report” may specify mandatory segments such as Result Table, Clinical Summary, Variant Details, Patient Demographics, and Methodology Section. A template of type “Clinical Chemistry Results” may specify a different set.

Upon receipt of an incoming clinical report and its template identifier, anomaly detection module 216 may retrieve the corresponding segment list and execute a segment presence check. For each expected segment, anomaly detection module 216 may apply a combination of structural heuristics and pattern-matching rules to determine whether that segment is locatable within the clinical report. Detection strategies may include positional scanning for section headings at expected layout coordinates, keyword and regular expression matching against segment header labels, and structural analysis of recurring table or block patterns associated with each segment type. Each segment may be assigned a binary presence flag (present or absent) based on the outcome of these checks.

Anomaly detection module 216 may aggregate the per-segment presence flags present_i and importance weights weight into a weighted completeness score Score, computed as the sum of the importance weights of all detected segments divided by the sum of the confidence weights of all expected segments. A formal representation of an example is:

Score=Σ(weight_i×present_i)/Σ(weight_i),

Where present_i is 1 if segment i was detected and 0 otherwise. This score ranges from 0 (no expected segments detected) to 1 (all expected segments detected). A configurable threshold, set per template type, governs the acceptance criterion: clinical reports scoring at or above the threshold proceed normally to the extraction pipeline, clinical reports scoring below the threshold are flagged as anomalous.

When the completeness score falls below the configured threshold, anomaly detection module 216 may generate a structured anomaly alert record. This record may include for example: (i) the resolved template identifier and the clinical report identifier; (ii) the list of specific segment names that were expected but not detected; (iii) the computed completeness score and the applicable threshold; (iv) an extraction confidence classification (e.g., LOW, VERY LOW) derived from the score magnitude; and (v) a recommended action, typically indicating that the report template should be reviewed and, if the structural change is confirmed, the pipeline instructions should be generated for that report type, e.g., by onboarding module 210. The anomaly alert may be delivered to a human-in-the-loop interface for operator review, and the corresponding template identifier may be added to a retraining queue, suspending automated extraction for that clinical report until the alert is resolved.

The alert record may be presented to an authorized operator through the human-in-the-loop interface, which may present (e.g., via a graphical user interface (GUI) on output devices 140) the list of missing segments, the reduced confidence score, and the recommended action in a structured review panel. The operator may elect to: (a) confirm the template change and initiate a retraining workflow for the affected report template; (b) override the alert and proceed with extraction at reduced confidence if the missing segments are deemed non-critical for the specific use case; or (c) reject the clinical report and return it to the submitter for re-submission with the correct template. All operator decisions are logged with a timestamp and operator identifier for audit purposes. Clinical reports flagged by anomaly detection module 216 may be provided to onboarding module 210, together with a set of labeled reports for generating pipeline instructions for that report template.

Segmentation module 218 may segment the clinical report into segments (e.g., logical units) using the report template. Segmentation may be performed using LLM of one or more LLMs 240 and/or regular expression-based extraction techniques. Segmentation may include identifying specific segments of the incoming clinical report, such as patient demographics, test results, or clinical interpretations. Each identified segment is stored in a dedicated repository (not shown), where it remains accessible for targeted analysis in the subsequent stages of the extraction and validation process.

The segments may serve as units of analysis for subsequent extraction operations. For example, extraction module 220 may perform extraction separately on each segment in the incoming clinical report. Performing extraction at the segment level may dramatically improve extraction precision and reduce hallucination risk. Segmentation module 218 may employ a dual-strategy detection mechanism, combining a rule-based regular expression detector with an LLM-based semantic detector, whose outputs are fused (e.g., combined) and validated.

Prior to segment detection, segmentation module 218 may perform a pre-processing pass that includes text layer extraction, layout normalization, and page boundary detection. Text is extracted while preserving positional metadata (e.g., bounding box coordinates, page number, font metrics) to support position-aware detection strategies. The layout of the incoming clinical report may be normalized to a canonical representation, referred to as the normalized text, in which header, footer, and body regions are distinguished, and multi-column layouts are linearized into a reading-order text stream. Page break positions may be recorded and made available to the segment detectors.

Segmentation module 218 may segment the incoming clinical report using the report template included in the pipeline instructions of the clinical report format (retrieved by pipeline instructions retrieval module 214). For each expected segment, the report format may specify a canonical segment name (e.g., Results Table, Clinical Summary, Variants Details, Patient Demographics), one or more heading keyword patterns used by the regex detector, positional priors indicating the typical location of the segment within a clinical report of that template (e.g., top third, bottom half), and a minimum confidence threshold below which an LLM segment detection is not accepted.

Segmentation module 218 may include a regex detector. The regex detector may apply the set of regular expression boundary rules (e.g., regular expression boundary rules generated by onboarding module 210) to the normalized text. For each expected segment, the regex detector may execute the following checks: (i) heading keyword matching, in which the text is scanned for exact or fuzzy matches against the set of known section heading labels associated with that segment type; (ii) positional boundary detection, in which structural markers such as horizontal rules, blank line clusters, or font-size transitions are used to identify candidate segment boundaries; and (iii) table and list structure recognition, in which recurring delimiter patterns are used to locate tabular data blocks characteristic of specific segment types. Each regex match may produce a candidate segment record containing the matched segment name, the start and end character offsets, and a pattern-match confidence score.

Segmentation module 218 may include an LLM segment detector. The LLM detector may be invoked in parallel with the regex detector and operate on fixed-length text windows across the normalized text of the clinical report. For each window, the LLM detector may submit an LLM segmentation prompt to an LLM of one or more LLMs 240, requesting the LLM to identify whether the window contains the beginning of a known segment type and, if so, to name the segment and assign a confidence score. The segmentation prompt may include few-shot examples drawn from the report template to anchor the LLM's predictions to the specific template. The LLM detector may be particularly effective for segments whose headings are absent, abbreviated, or formatted inconsistently across instances of the clinical report. Each LLM response produces a candidate segment record in the same schema as the regex detector output, enabling uniform downstream fusion.

Segmentation module 218 may combine, fuse or unify the candidate segments produced by the regex detector and LLM detector. Combining candidate segments produced by the regex and LLM detectors may include merging overlapping or conflicting candidate segments into a single set of detected segments. Combining may be proceeded by grouping candidate segments that refer to the same segment type, comparing their boundary offsets and confidence scores, and selecting the candidate with the higher confidence as the representative detection. Where both detectors agree on a segment boundary within a configurable tolerance, their confidence scores are combined using a weighted sum to produce an elevated composite confidence. Candidates that are detected by only one detector and fall below a minimum confidence threshold are discarded. Combining candidate segments provides an ordered list of non-overlapping segment detections covering the clinical report.

Segmentation module 218 may validate segment boundaries. The fused segment list is subjected to a boundary validation pass that verifies the structural integrity of the detected segmentation. This pass checks that no two detected segments overlap in their character offset ranges; that no gap larger than a configurable threshold exists between consecutive segments; and that each detected segment is assigned a unique segment identifier composed of an identifier of the report format, the segment type name, and a sequence number. Segments failing validation are flagged and either re-detected with relaxed parameters or escalated to anomaly detection module 216.

Each validated segment may be stored as a structured record in a segment repository (not shown). The record contains: the unique segment identifier, the segment type label, the start and end character offsets within the normalized text, the verbatim source text of the segment, the detection strategy that produced the final boundary (regex, LLM, or fused), and the composite confidence score. These records are the primary input used by extraction module 220, which may apply targeted LLM extraction prompts to each segment independently to perform high-precision data field-level data extraction.

Extraction module 220 may extract data from each segment by applying at least one extraction prompt and regular expressions (regex), that are specific to the segment, to an LLM of one or more LLMs 240. By restricting the focus of the LLM to text segments that are smaller than the clinical report and have limited context, the accuracy of the extraction may significantly improve relatively to extracting data from the full clinical report. The LLM prompts may be designated to extract only the minimal, specific pieces of data present in the target segment, thereby maximizing accuracy and meeting medical-grade data standards. The data extracted by extraction module 220 form the raw extracted data.

Extraction module 220 may extract data from each segment in turn, by providing the source text of the segment and the extraction prompts associated with the segment in the pipeline instructions to the relevant model from one or more LLMs 240.

For each segment, extraction module 220 may execute the extraction prompt by inserting the segment's source text into the designated input placeholder, adding the data field names from the output schema so the LLM has an explicit specification of what to extract, and appending few-shot extraction examples drawn from the configuration to demonstrate the expected output format. The extraction prompt may instruct the LLM to return its response as a structured JSON object conforming to the output schema, with each data field name as a key.

The extraction prompt may be input or submitted to the LLM that may process the segment text within the context of the extraction prompt and return the raw extracted data, e.g., in JSON format. Extraction module 220 may record the raw extracted data together with any data confidence signals returned by the LLM. If the response of the LLM is malformed or fails to parse as valid JSON, a retry may be issued with an amended extraction prompt that reinforces the output format constraint. A maximum retry count may be enforced; exhaustion of retries may cause the segment to be flagged for human-in-the-loop review.

An example extraction prompt, as generated by onboarding module 210 and used by extraction module 220, is provided below:
Role
You are a medical data extractor specialized in parsing genetic test reports in PDF format.
Objective
Your task is to extract structured information from the genetic report, including:
   1 Patient details
   2 Sample/specimen information
   3 Positive genetic findings
   4 FMR1 (Fragile X Syndrome) screening results
Output Format
Return the results strictly as a JSON object, using the following structure:

```
{
  “findings”: [
    {
      “variant”: “mutation_name”,
      “gene”: “gene_name”,
      “state”: “state”,
      “proteinName”: “protein_name”,
      “clinicalSignificance”: “pathogenic”
    }
  ],
  “fmr”: {
    “allele1”: 29,
    “allele2”: 29,
    “state”: “Normal”
  },
  “specimenId”: “specimen_id”,
  “patient”: {
    “firstName”: “first_name”,
    “lastName”: “last_name”,
    “DOB”: “date_of_birth”
  }
}
```

Extraction Rules
Patient & Specimen
   Extract the patient's first name, last name, and date of birth.
   Extract the specimen ID from the first page of the report.
Findings
   Extract only positive findings (e.g., carrier or pathogenic variants).
   Each finding must include:
   variant: mutation name (max 2 tokens, e.g., c.1244A>G)
   gene: associated gene name (e.g., CFTR)
   state: interpretation status (e.g., carrier, affected, etc.)
   proteinName: format like p.Ala327Pro (remove parentheses if present)
   clinicalSignificance: value based on lab classification—prioritize lab's classification over ClinVar
Examples of valid proteinName: [symbol] (p.Arg492Trp)→p.Arg492Trp [symbol] (p.Met34Thr)→p.Met34Thr
Special case rule: [symbol] If the variant includes phrases like -alpha3.7 deletion (-alpha/alpha alpha), extract only the main part: -alpha3.7 deletion.
Clinical significance determination: [symbol] Use the lab's classification when available. It is usually expressed in a sentence such as:
“The laboratory classifies this variant as pathogenic.” [symbol] Return the clinicalSignificance as the exact classification term (e.g., “pathogenic”, “likely pathogenic”).
FMR1 Results
   Located under the findings table in a section like:
“The repeat sizes detected were: 29 and 29 repeats. . . . . These results are within the normal range.”
   Extract:
   allele1: first repeat count (e.g., 29)
   allele2: second repeat count (e.g., 29)
   state: interpretation (Normal, Premutation, Full mutation, etc.)
Important Notes.
   Return only the JSON object—no explanations, no text, no markdown, no comments.
   Follow the exact property names and structure in the output format.

```
Example Output
{
  “findings”: [
    {
      “variant”: “c.1244A>G”,
      “gene”: “CFTR”,
      “state”: “carrier”,
      “proteinName”: “p.Ala327Pro”,
      “clinicalSignificance”: “pathogenic”
    }
  ],
  “fmr”: {
    “allele1”: 29,
    “allele2”: 29,
    “state”: “Normal”
  },
  “specimenId”: “124-129-1742-0”,
  “patient”: {
    “firstName”: “John”,
    “lastName”: “Doe”,
    “DOB”: “2000-06-01”
  }
}
```

API input and output examples are provided below:

```
Input example (preconception, PRECO):
{
  “organizationId”: 3,
  “file”: “s3...”,
  “clinicalIndication”: “PRECO”,
  “offeringId”: 45,
  “extractionType”: [
    “SAMPLE_DETAILS”,
    “PATIENT_DETAILS”,
    “FINDINGS”,
    “DISEASE_DESCRIPTION”
  ],
  “customPayload”: { }
}
  Output example (PRECO):
{
  “patient”: {
    “firstName”: “John”,
    “lastName”: “Smith”,
    “DOB”: “1/1/1990”
  },
  “sampleNumber”: “BLD0334-01”,
  “findings”: [
    {
      “gene”: “IDUA”,
      “variant”: “c.979G>C”,
      “protein”: “p.Ala327Pro”,
      “clinicalSignificance”: “pathogenic”,
      “state”: “CARRIER”,
      “soruce”: “IDUA, c.979G>C,(p.Ala327Pro), Pathogenic”,
      “guards”: [
        {
          “guard”: “gene”,
          “calls”: [“Non valid gene”],
          “confidence”: 0.1
        }
      ]
    }
  ],
  “FMR”: {
    “allele1”: 55,
    “allele2”: 100,
    “state”: “Intermediate”,
    “guards”: [
      {
        “guard”: “gene”,
        “calls”: [“Non valid gene”],
        “confidence”: 0.1
      }
    ]
  },
  “confidenceScore”: 0,
  “guards”: [
    {
      “guard”: “findingsGuard”,
      “calls”: [“Missing finding: CFTR:c.123C>T”]
    }
  ]
}
```

Segment guard validation module 222 and template guard validation module 224 may evaluate the data extracted by extraction module 220 against guard rules. The guard rules may be segment specific or template specific and may be generated by onboarding module 210 as part of the onboarding process. The guard rules may be a part of the pipeline instructions generated by onboarding module 210, stored in pipeline instructions repository 132 and retrieved by pipeline instructions retrieval module 214. The guard rules may include: presence guards that verify that all data fields marked as mandatory in the pipeline instructions are populated with non-null values in the extracted data, range guard rules that verify that numeric data in the extracted data fall within clinically plausible ranges defined in the pipeline instructions, and format guard rules that verify that string data fields in the extracted data conform to expected patterns.

The guard rule types may include for example:
LLM-based guard rules that include an evaluation prompt including the extracted data and the segment from which the extracted data were extracted as context.
Code-based guard rules that apply logic rules to the extracted data.
Regex-based guard rules that validate format of extracted string data fields within the data by matching the extracted string data fields against one or more regular expression patterns.
Rule-based guard rules that evaluate the extracted data against domain knowledge bases.
Generic guard rules that validate generic data fields such as date of birth (DOB), patient name and general results.
Clinical indication-specific guard rules that validate variant names and gene names.

An extracted data item that satisfies all applicable guard rules may be considered as a validated data item with a PASS guard status. An extracted data item that fails one or more guard rules may be subjected to re-extraction, e.g., extraction module 220 may re-invoke the LLM with a refined prompt that may explicitly identify the failing fields and requests correction. If the re-extraction also fails the guard rule, the data item may be emitted from further analysis and sent with a FAIL guard status and a populated failure detail list for human-in-the-loop review.

Segment guard validation module 222 may validate the extracted data items extracted by extraction module 220 against segment guard rules. Segment guard validation module 222 may calculate the confidence level of the extracted data items of a segment. For example, if extracted data item contains variant name then a set of segment guard rules may verify that the variant name complies with the variant name nomenclature. Segment guard validation module 222 may generate an extracted data record for each validated data item. The extracted data record may include the segment identifier, the set of data name-to-value pairs extracted from that segment, a composite confidence score for the extracted data item (combining LLM confidence and supplementary code agreement), and the guard status with any associated failure detail. Once all segments in the clinical report have been processed, the individual extracted data records may be assembled by segment guard validation module 222 into a report-level final output report. This report may map segment names to their corresponding extracted data record and constitute the primary structured data output produced by system 200.

Template guard validation module 224 may include a central service that coordinates the application of template guard rules. Template guard validation module 224 may validate extracted data items against template guard rules. Each template guard rule may evaluate the extracted data items, provide a confidence score, and return evaluation callouts and comments.

Template guard validation module 224 may validate the extracted data items extracted by extraction module 220 and validated by segment guard validation module 222 against template guard rules. The template guard rules may be designed to assess a specific dimension of extraction quality. Template guard validation module 224 may provide a guard evaluation report that may quantify the overall extraction confidence and surface specific data field-level issues for system 200 or for human review.

The template guard rules may be a part of the pipeline instructions generated by onboarding module 210 or manually, stored in pipeline instruction repository 132 and retrieved by pipeline instructions retrieval module 214. Each template guard rule record in the pipeline instructions may include an identifier, an implementation type (e.g., LLM-based, code-based, regex-based, rule-based, or a combination thereof), the extraction data fields and segments the rule targets, a relative weight in the aggregated confidence score, and an order in which the guard rule should be evaluated relative to other guard rules. Some template guard rules may be universal across all templates (e.g., generic guards such as patient name and date-of-birth validators) and some guards may be specific to a clinical domain or report format (e.g., clinical indication guards, such as variant name and gene name validators for genomic reports).

Upon receipt of extracted data items, template guard validation module 224 may retrieve the template guard rules from the pipeline instructions. Template guard validation module 224 may route the relevant portions of the validated data items to each template guard rule in accordance with an evaluation order. Template guard rules that are independent of one another may be evaluated in parallel to minimize latency. Template guard rules that depend on the output of a preceding template guard rules are evaluated sequentially in the order of dependencies.

The template guard rules may include LLM-based template guard rules that include a structured evaluation prompt including the extracted data items and the original segment source text as context. The evaluation prompt may instruct the LLM (of one or more LLMs 240) to assess the accuracy and completeness of the extracted data items against the source text, to identify any hallucinated, missing, or incorrectly formatted values, and to return a confidence score together with specific callouts for each identified issue. The LLM-based template guard rules may be effective for evaluating free-text data fields, semantic coherence across related data fields, and complex clinical findings where rule-based validation is insufficient. An example LLM guard is the General Results Assessment Guard, which evaluates the accuracy and completeness of extracted genetic findings by comparing the structured extraction against the source text of the Results segment.

The template guard rules may include code-based guard rules. The code-based guard rules may apply deterministic programmatic logic to the extracted data items. Code-based guard rules may include: date validity checks (e.g., verifying that a date of birth is a valid calendar date that falls within a plausible range); numeric range validation (e.g., verifying that a reported laboratory extracted data value falls within a defined clinical reference interval); cross-field consistency verification (e.g., confirming that a reported age is consistent with the reported date of birth and the report date); and sample identifier format verification. The code-based guard rules may execute in microseconds and produce deterministic, auditable results, making them the preferred implementation for any validation criterion that can be precisely expressed as a computational predicate.

The template guard rules may include regex-based guard rules. The regex-based guard rules may validate the format and structure of extracted data items by matching the extracted data items against one or more regular expression patterns. Each regex-based guard rule may be associated with a named data field or set of data fields and a pattern library specific to the clinical report format. A match against the expected pattern yields a full or partial confidence contribution depending on a strictness tier of the pattern. A non-match reduces the confidence contribution of the extracted data items and generates a callout specifying the nature of the format violation. Regex-based guard rules may validate extracted data items such as variant nomenclature against human genome variation society (HGVS) notation patterns, validate clinical codes against international classification of diseases (ICD) or current procedural terminology (CPT) code format patterns, validate patient identifiers against institution-specific identifier schemas, etc.

The template guard rules may include rule-based guard rules. The rule-based guard rules may evaluate extracted data items against curated domain knowledge bases. A rule-based guard rule may be configured with a reference set appropriate to the field the rule targets, for example, a gene name rule-based guard rule may be configured with the set of valid gene names included in the specific genetic test panel associated with the template. If the extracted value appears in the reference set, the rule-based guard rule may assign a high confidence score. If the value is absent from the reference set, the rule-based guard rule may assign a zero or reduced confidence score and generate a callout identifying the value as outside the expected vocabulary. Rule-based guard rules can also encode clinical logic, such as verifying that a reported pathogenicity classification is consistent with the variant classification framework specified by the template (e.g., the American college of medical genetics and genomics and the association for molecular pathology (ACMG/AMP) criteria tiers).

Each template guard rule may produce a result record containing a numeric confidence score (e.g., in the range [0.0, 1.0]), a status classification (e.g., PASS, WARN, or FAIL), and a list of human-readable callouts and comments describing any issues identified. The template guard validation module 224 may combine the individual guard scores into an overall extraction confidence level, e.g., using a weighted average, where the weight of each template guard rule may be specified in the pipeline instructions. The resulting aggregate score may be mapped to a discrete confidence band, e.g., HIGH (score≥0.85), MEDIUM (0.60-0.85), LOW (0.35-0.60), or VERY LOW (score<0.35). This confidence level may be included in a guard evaluation report alongside the per-guard scores, statuses, and callouts and may be the output of template guard validation module 224. The guard evaluation report may include the overall confidence level, the weighted aggregate score, and the complete set of per template guard rule result records. Clinical reports achieving a HIGH or MEDIUM confidence level may be forwarded for final assembly and delivery. Clinical reports classified as LOW confidence are flagged with a warning and forwarded with the guard evaluation report attached, enabling downstream consumers to apply additional scrutiny. Clinical reports classified as VERY LOW confidence trigger a human-in-the-loop review notification, suspending automated delivery until an operator reviews the extraction, the guard callouts, and either approves the result, initiates a targeted re-extraction, or rejects the clinical report.

The segment and template guard rules may include generic guard rules that may apply to all report formats, such as verifying patient name, date of birth, and sample number:
Date of birth guard rule that evaluates validity of the date of birth and provides score accordingly.
Patient name guard rule that evaluates the validity of the patient first name and last name and provides score accordingly.
General results assessment rule that uses an LLM from one or more LLMs 240 to evaluate the accuracy and completeness of the extracts genetic findings, as in the following prompt example:
Role
You are a medical data extractor specialized in parsing genetic test reports in PDF format.
Objective
Your task is to extract structured information from the genetic report, including:
   1 Patient details
   2 Sample/specimen information
   3 Positive genetic findings
   4 FMR1 (Fragile X Syndrome) screening results
Output Format
Return the results strictly as a JSON object, using the following structure:

```
{
  “findings”: [
    {
      “variant”: “mutation_name”,
      “gene”: “gene_name”,
      “state”: “state”,
      “proteinName”: “protein_name”,
      “clinicalSignificance”: “pathogenic”
    }
  ],
  “fmr”: {
    “allele1”: 29,
    “allele2”: 29,
    “state”: “Normal”
  },
  “specimenId”: “specimen_id”,
  “patient”: {
    “firstName”: “first_name”,
    “lastName”: “last_name”,
    “DOB”: “date_of_birth”
  }
}
```

Extraction Rules
Patient & Specimen
   Extract the patient's first name, last name, and date of birth.
   Extract the specimen ID from the first page of the report.
Findings
   Extract only positive findings (e.g., carrier or pathogenic variants).
   Each finding must include:
      variant: mutation name (max 2 tokens, e.g., c.1244A>G)
      gene: associated gene name (e.g., CFTR)
      state: interpretation status (e.g., carrier, affected, etc.)
      proteinName: format like p.Ala327Pro (remove parentheses if present)
      clinicalSignificance: value based on lab classification—prioritize lab's classification over Clin Var
Examples of valid proteinName: [symbol] (p.Arg492Trp)→p.Arg492Trp [symbol] (p.Met34Thr)→p.Met34Thr
Special case rule: [symbol] If the variant includes phrases like-alpha3.7 deletion (-alpha/alpha alpha), extract only the main part: -alpha3.7 deletion.
Clinical significance determination: [symbol] Use the lab's classification when available. It is usually expressed in a sentence such as:
“The laboratory classifies this variant as pathogenic.” [symbol] Return the clinicalSignificance as the exact classification term (e.g., “pathogenic”, “likely pathogenic”).
FMR1 Results
   Located under the findings table in a section like:
“The repeat sizes detected were: 29 and 29 repeats. . . . . These results are within the normal range.”
   Extract:
   allele1: first repeat count (e.g., 29)
   allele2: second repeat count (e.g., 29)
   state: interpretation (Normal, Premutation, Full mutation, etc.)
Important Notes
   Return only the JSON object-no explanations, no text, no markdown, no comments.
   Follow the exact property names and structure in the output format.
Example Output

```
{
  “findings”: [
    {
      “variant”: “c.1244A>G”,
      “gene”: “CFTR”,
      “state”: “carrier”,
      “proteinName”: “p.Ala327Pro”,
      “clinicalSignificance”: “pathogenic”
    }
  ],
  “fmr”: {
    “allele1”: 29,
    “allele2”: 29,
    “state”: “Normal”
  },
  “specimenId”: “124-129-1742-0”,
  “patient”: {
    “firstName”: “John”,
    “lastName”: “Doe”,
    “DOB”: “2000-06-01”
  }
}
```

The segment and template guard rules may include clinical indication-specific guard rules. Clinical indication-specific guard rules may include logic tailored to verify extracted data in a specific clinical domain (e.g., expanded carrier screening, non-invasive prenatal screening, oncology, etc.). Example clinical indication-specific guard rules for verifying gene panel inclusion and validating result structure may include:
A variant name guard rule that evaluates an extracted variant name against a set of predefined variant name patterns and assigns a confidence score accordingly. If the variant name does not match or only partially matches any of the expected patterns, the variant name guard rule may adjust the confidence score appropriately and may add a relevant comment, as demonstrated in the following example:

```
...
“variant”: “c.979G>C[ ]tttt”,
“guards” : [
    {
      “guard”: “variantName”,
      “calls” :[“Variant name contains unexpected characters”],
      “score” : 0.2
      }
  ]
...
```

A gene name guard rule that may evaluate the gene name based on a predefined list of valid gene names relevant to the specific test. If the extracted gene name is not in the list, the gene name guard rule may set the confidence score of the extracted data to a low confidence level such as 0 and may provide a comment indicating that the gene name is not part of the valid set for this test.

Results aggregator 226 may collect and merge outputs from various modules including guard results and confidence scores into a final result object for downstream use. The following example demonstrates the output of results aggregator 226:

```
{
  “patient”: {
      “firstName”: “John”,
      “lastName”: “Smith”,
      “DOB”: “1/1/1990”
  },
  “sampleNumber”: “BLD0334-01”,
  “findings”: [
      {
        “gene”: “IDUA”,
        “variant”: “c.979G>C”,
        “protein”: “p.Ala327Pro”,
        “clinicalSignificance”: “pathogenic”,
        “state”: “CARRIER”,
        “guards” : [
          {
            “guard”: “gene”,
            “calls” :[“Invalid gene IDUA”],
            “score” : 0.0
            }
        ]
      },
  ],
  “FMR” : {
    “allele1” : 55,
    “allele2” : 100,
    “state” : “Intermediate”,
    “guards” : [
          {
            “guard”: “allele number”,
            “calls”: [“Illegal allele repeat”],
            “score” : 0.0
            }
        ]
  },
  “score”: 0,
  “guards”: [ {
              “guard” : “findingsGuard”,
              “calls” : [“Missing finding: CFTR:c.123C>T”],
              “score” : 0
              }
  ]
}
```

Post processing module 228 may receive operator corrections made to previously extracted data from user feedback module 230, detect, using one or more LLMs 240, patterns in the operator corrections, generate, using the one or more LLMs 240, transformation rules that encode the patterns in the operator corrections into the transformation rules, and apply the transformation rules to the extracted data to obtain normalized data. Post processing module 228 may apply transformation rules that target non-overlapping extracted data in parallel to each other (e.g. substantially at the same time or overlapping in time) and transformation rules that target the same extracted data sequentially (e.g. one after another in time; not in parallel). The transformation rule types may include code transformation rules that encode transformation logic as executable software code, regular expressions (regex) transformation rules that apply regular expression patterns to the data, prompt transformation rules that comprise an LLM prompt that describes the transformation rule, LLM-generated rules that are produced by an LLM rule synthesizer and encode transformation patterns inferred from operator correction behavior.

Post processing module 228 may include a high-level orchestration layer that operates on the complete set of data gathered from the individual extraction segments. Post processing module 228 may aggregate results from all previously processed segments to identify cross-report patterns or contradictions that individual segments might miss, may employ a combination of deterministic business rules and LLM-based reasoning to refine and standardize the extraction, may apply advanced clinical logic that goes beyond simple data recovery, providing clinical interpretation and context to the structured findings (e.g., correlating specific gene variants with overall diagnostic impressions) and may improve the extraction results, ensure that the final output meets the rigorous standards required for medical-grade clinical data.

Post processing module 228 may include a rule-driven transformation layer that operates on the data extracted by extraction module 220, together with the guard evaluation report generated by the segment guard validation module 222 and template guard validation module 224. Whereas extraction module 220 may focus on extracting structured data from individual segments of the clinical report, post processing module 228 may apply transformation rules such as normalization, standardization, and semantic enrichment rules, across the complete set of extracted data, ensuring that the final output report meets the data-quality requirements of downstream clinical and analytical systems. The transformation rules may be part of the pipeline instructions stored in pipeline instructions repository 132 and retrieved by pipeline instructions retrieval module 214. Each transformation rule may be associated with a specific report template identifier and optionally with a specific segment, allowing per-template control over which transformation rules are applied to which data fields. Some transformation rules may encode deterministic transformations such as unit normalization (e.g. mg/dL to mmol/L), date format standardization (e.g. DD/MM/YYYY to ISO 8601), controlled vocabulary mapping (e.g. gene symbol aliases to canonical HGNC identifiers), and type coercions (e.g. free-text numeric strings to typed floating-point values). Some transformation rules may be generated manually by domain engineers. Some transformation rules may be LLM-generated rules generated by onboarding module 210 or by refinement module 232 from patterns detected in operator correction events.

The transformation rules may include for example code rules, regex rules, LLM rules, and LLM-generated rules, or other rules. Post processing module 228 may construct a transformation plan that specifies, for each extracted data, the sequence of transformation rules to be applied and the order of execution. Transformation rules targeting non-overlapping data may be executed in parallel to minimize processing latency, while transformation rules targeting the same data may be executed in a deterministic serial order defined by a rule priority attribute included in the pipeline instructions.

Post processing module 228 may support several (e.g four) distinct transformation rule execution lanes, each implementing a different transformation mechanism: (i) code rules encode transformation logic as executable Python functions; they perform operations such as format standardization, numeric unit conversion, and type coercion with full programmatic control and deterministic output. (ii) regex rules apply compiled regular expression patterns to data values; they perform pattern-based extraction, string reformatting, token isolation, and format validation. (iii) LLM rules delegate transformation decisions to an LLM via a structured prompt; they are used for semantically complex operations such as clinical terminology normalization, cross-field inference, and conversion of free text to structured values, where rule-based approaches are insufficient. (iv) LLM-generated rules may be structurally identical to code rules but are produced by onboarding module 210 rather than authored by engineers; they encode transformation patterns inferred from operator correction behavior. The LLM-generated rules may be subject to a mandatory review and versioning process before deployment.

Following invocation of the LLM of one or more LLMs 240, post processing module 228 may apply the transformation rules to the raw extracted data. This step may serve four purposes. First, regex patterns specific to the segment type may be applied to the original segment source text to extract or validate individual data values that the LLM of one or more LLMs 240 may have missed, mis-formatted, or hallucinated. Where a regex extraction contradicts output of the LLM of one or more LLMs 240, the regex result (e.g., data extracted using regex) is preferred for data fields covered by a high-precision pattern. Second, unit normalization functions may be applied to convert extracted numeric values to a canonical unit system (e.g., converting mg/dL to mmol/L for glucose). Third, some transformation rules may transform extracted string values to their target types as specified in the output schema (e.g., numeric, date, Boolean, enumerated). Fourth, some transformation rules may execute cross-data field consistency checks within the segment to detect internal contradictions and flag them for guard processing.

Post processing module 228 may generate transformation rules from operator correction patterns. Generating transformation rules from operator correction patterns by post processing module 228 may transform recurring operator corrections, e.g., manual edits made by authorized human reviewers to extracted data previously provided to the authorized human reviewers, into executable transformation rules. Generating transformation rules from operator correction patterns and applying the generated transformation rules to extracted data may improve the technology of medical data extraction by encoding expert domain knowledge as durable system behavior and improve the precision of the extracted data. Generating transformation rules from operator correction patterns automatically as disclosed herein may achieve this improvement without requiring manual engineering effort.

Post processing module 228 may analyze the correction history of data extracted from a complete clinical report, examine operator edits across all data items and segments of a clinical report, and draw inferences from the combination of one or mor corrections. This may enable detecting systemic transformation patterns that include a single data item as well as corrections that span over several data items or segments, patterns that would remain invisible when examining any single field correction in isolation. For each correction, post processing module 228 may generate a structured record containing: the template identifier (an identified of the clinical report format), the segment, the data field name, the original extracted data item value, the operator-corrected value, and a timestamp. Post processing module 228 may retrieve these records grouped by clinical report format and aggregated across all fields of each processed clinical report, enabling pattern analysis at the full clinical report extraction level rather than in isolation.

Post processing module 228 may periodically retrieve batches of correction records. Post processing module 228 may provide an LLM of one or more LLMs 240 with the complete correction context of a clinical report, the full set of operator corrections applied across all segments and fields, enabling the LLM to reason across multiple findings simultaneously and infer cross-field dependencies and compound transformation patterns. Post processing module 228 may generate a structured prompt presenting the aggregated correction evidence and instruct the LLM to identify the underlying transformation logic. For example, inferring that a correction to a reference range data field consistently co-occurs with a correction to a corresponding unit data field, or that specific combinations of findings across multiple segments systematically imply a different clinical interpretation of the report as a whole. The LLM may generate a candidate transformation rule, e.g., as an executable Python function, together with a natural-language explanation of the inferred transformation logic, the set of template and data field selectors to which the rule should apply, and a confidence score reflecting the consistency and strength of the observed pattern across the correction sample. In some embodiments, the generated transformation rules may be reviewed by an authorized operator.

Post processing module 228 may apply the transformation rules to the associated extracted data items. Transformation rules that refer to overlapping extracted data items may be applied sequentially, where each transformation rule receives the current data value and returns a transformed data value, which becomes the input to the next transformation rule in the sequence. Post processing module 228 may maintain a transformation audit trail for each data item, recording the rule identifier, rule type, input value, output value, and execution timestamp for every transformation rule. This audit trail may be included in a final output report and provide a complete, reproducible account of how each final data value was derived from the raw extraction data.

Post processing module 228 may mark data items for which all applicable transformation rules executed successfully as normalized data items. Data items for which one or more transformation rules raise a transformation error may be flagged with an error annotation and retain the pre-transformation value, so that transformation rule failures may not result in data loss. Post processing module 228 may review the guard evaluation report produced by segment guard validation module 222 and template guard validation module 224 prior to applying the transformation rules. Data items with a VERY LOW confidence rating from the guard evaluation may optionally bypass execution of the transformation rules to avoid compounding uncertain extractions with further transformations.

Upon applying all applicable transformation rules associated with a clinical report format to the extracted data, post processing module 228 may generate a post-processing report that may include the normalized data values, the transformation audit trail, updated confidence annotations reflecting any changes introduced by post-processing, and a summary of any data items that were flagged during rule execution. The post-processing report may be included in the final output report. The complete transformation audit trail embedded in the output enables downstream consumers to reproduce any field value, inspect the rule chain that produced it, and attribute corrections to specific rules, capabilities that are particularly important in regulated clinical environments where data provenance and auditability are mandatory requirements.

Refinement module 232 may refine the extracted data based on the evaluation made by segment guard validation module 222 and template guard validation module 224. Refinement module 232 may refine the extracted data in case the evaluating an extracted data item against a certain guard rule fails (e.g., one of the template guard rules or segment guard rules). Refining may include providing the extracted data item, the guard rule, the segment including the first extracted data item and the extraction prompt applied to the at least one LLM to extract the first extracted data item, to an LLM of one or more LLMs 240, updating, by the LLM of one or more LLMs 240, the extraction prompt applied to the LLM of one or more LLMs 240 to extract the first extracted data item, and applying the updated extraction prompt to the segment including the extracted data item to obtain a refined extracted data item. For cases of semantic mismatch and missing value failure modes refining may include updating the extraction prompt used to extract the extracted data item to include a guard failure reason, constraining the extracted data to the expected output format, and applying the updated extraction prompt (e.g., by extraction module 220) to the segment including the first extracted data item to extract a refined first data item from the updated segment. For missing-value failures refining may include repeating segmenting the clinical report into segments (e.g., by segmentation module 218) with adjusted boundary parameters, and, for the updated segment including the first extracted data item, repeating applying the extraction prompt (e.g., by extraction module 220) to the at least one LLM to extract a refined first data item from the updated segment. For data that follow deterministic patterns refining may include applying regex rules to the segment including the first extracted data item (e.g., by extraction module 220) to extract a refined first data item from the updated segment. For complex segments that contain multiple overlapping data items, refining may include segmenting the segment including the first extracted data item into smaller, non-overlapping sub-segments (e.g., by segmentation module 218), applying independent extraction prompts to each sub-segment (e.g., by extraction module 220) and merging the resulting data to extract a refined data item from the updated segment.

Refinement module 232 may be an orchestration component responsible for performing a refinement service that evaluates outputs of segment guard validation module 222 and template guard validation module 224 and decide, at two distinct checkpoints, whether the extracted data meet a quality threshold required for downstream use or whether targeted re-processing is warranted. Refinement module 232 may implement a structured, two-phase decision cycle: phase 1 that operates on the raw extracted data before being processed by post processing module 228, and phase 2 that operates on the normalized extracted data (e.g., the extracted data after being processed by post processing module 228). By separating these two evaluation points, refinement module 232 may ensure that guard-driven refinement addresses both deficiencies in the raw extracted data (e.g., data as extracted by extraction module 220) and deficiencies in the normalized extracted data (e.g., data after being transformed by post processing module 228) independently, with each phase invoking its own dedicated refinement service.

Refinement module 232 may trigger phase 1 of the refinement service after extraction module 220 provides the raw extracted data of a whole examiner clinical report, before post processing module 228 is invoked. The applicable segment and template guard rules may be evaluated against the raw extracted data (e.g., by segment guard validation module 222 and template guard validation module 224), producing a phase 1 guard evaluation report including per-data item confidence scores, anomaly indicators, and mandatory-field presence flags. Refinement module 232 may use the phase 1 guard evaluation for determining a refinement policy.

If all confidence scores meet or exceed the template-defined acceptance threshold, refinement module 232 may issue a PASS decision and forward the raw extracted data directly to post processing module 228 without modification. If, however, one or more guards confidence scores do not meet or exceed the template-defined acceptance threshold, refinement module 232 may issue a REFINE decision and provide the affected raw data items, together with the original segment of the analyzed clinical report, and the guard feedback, to a phase 1 refinement service of refinement module 232. The phase 1 refinement service may perform re-extraction of the flagged segments, typically by issuing a modified or more specific extraction prompt to one or more LLMs 240, or by invoking an alternative extraction strategy, and returns updated extracted data to the extraction output. The relevant guard rules may then be applied on the updated extracted data, and the routing decision is re-evaluated. A configurable maximum iteration count prevents infinite refinement loops. If the maximum is reached without achieving the acceptance threshold, the affected data items are annotated with a LOW CONFIDENCE flag and forwarded to post processing module 228 with an escalation indicator.

Refinement module 232 may perform phase 2 of the refinement service after post processing module 228 completes applying the relevant transformation rules. A second set of guard rules, the post-guard rules (that may also include segment and template guard rules), may be executed against the normalized, post-processed data (e.g., by segment guard validation module 222 and template guard validation module 224). Post-guard rules evaluate the quality of the normalized data rather than the raw extracted data, and are therefore designed to detect anomalies that arise specifically from the post-processing step, such as incorrect unit conversions, failed controlled-vocabulary mappings, or structural irregularities introduced by LLM-generated rules. The phase 2 guard evaluation report may record anomalies and post-normalization confidence scores for each field.

Refinement module 232 may apply the same routing logic in phase 2 as in phase 1 but operate in a different context: a REFINE decision in phase 2 dispatches the flagged data item to the phase 2 refinement service, which performs targeted re-application of the applicable transformation rule on the affected raw extracted data rather than re-extraction from the source clinical report. This may involve re-running a subset of transformation rules (e.g., by post processing module 228), invoking alternative normalization strategies, or escalating to an LLM-driven correction step. Updated data values are fed back through the phase 2 refinement service, and the routing decision is re-evaluated. As in phase 1, a maximum iteration guard caps the refinement loop. Fields that cannot be resolved within the maximum iteration budget are annotated with a LOW CONFIDENCE flag and routed to a human-review notification. The refined data may be considered the final extracted data and may be provided with appropriate annotations to the user, which may be a human operator or a computerized clinical and analytical systems, or store the final extracted data in a clinical report database.

Pre-guard rules and post-guard rules may have the same guard rules types used by segment guard validation module 222 and template guard validation module 224 (LLM guards, code guards, regex guards, and rule guards), but their configurations and evaluation targets may differ. Pre-guard rules may be configured to evaluate the faithfulness of the extracted data to the source clinical report, they check that mandatory segments are present, that extracted values fall within clinically plausible ranges, and that data field types match the expected schema. Post-guard rules may be configured to evaluate the semantic and structural correctness of the normalized data values, they verify that unit conversions produced values within expected ranges, that controlled vocabulary mappings resolved to valid canonical identifiers, and that field relationships preserved under normalization are internally consistent. The pre-guard rules and post-guard rules may be included in the pipeline instructions of the clinical report format. The full refinement history including the phase-1 and phase-2 guard evaluation reports, the number of refinement iterations performed in each phase, the identity of the guards that triggered each REFINE decision, and the final confidence annotations may be included in the final clinical data extraction report.

Refinement module 232 may include a refinement service that may apply additional LLM-based extraction passes based on guard feedback to refine specific segments of the extracted data, enabling adaptive correction and enhancement of the output.

A refinement service may be a targeted re-extraction component that is invoked whenever a guard rule reports a confidence score below the template-defined acceptance threshold for a specific segment or data item. Rather than re-processing the entire clinical report, the refinement service uses the guard feedback which identifies the data that failed, the guard rule that flagged the failure, and the nature of the failure, and applies one or more focused re-extraction strategies directed at the affected segment, leaving all other data items intact. The refinement service may be invoked independently in each of the two refinement phases, with phase 1 addressing deficiencies in the raw extracted data and phase 2 addressing deficiencies in the normalized data.

A refinement service may analyze the output of the guard rule originating the failure to extract three attributes from the report: the guard rule type that produced the low-confidence flag (LLM guard, code guard, regex guard, or rule guard); the failure reason, which is a structured or natural-language description of why the guard rejected the extracted value (for example, ‘numeric value outside clinically plausible range’, ‘gene symbol does not match canonical HGNC identifier’, or ‘mandatory field absent from extraction output’); and the failure mode classification, which categorizes the failure into one of a set of standard failure modes: missing value, wrong format, out-of-range value, semantic mismatch, or structural inconsistency. The failure mode classification may determine which re-extraction approach is most likely to correct the deficiency.

A refinement service may map the analyzed failure mode to one or more ordered re-extraction strategies. The refinement service may consult a strategy routing table, configured per clinical report format and per guard rule type, that specifies the preferred strategy sequence for each failure mode. Four re-extraction strategies may be supported: (i) refined LLM prompt—the refinement service re-invokes the segment extraction LLM with an augmented prompt that incorporates the guard failure reason as additional context, includes one or more few-shot correction examples derived from historical approved extractions, and explicitly constrains the expected output format; this strategy may be preferred for semantic mismatch and missing-value failure modes where the original prompt was insufficiently specific. (ii) alternative segment boundary—the refinement service may re-run the segment detection step with adjusted boundary parameters, expanding or shifting the detected segment region to capture content that may have been excluded by the original boundary heuristic, then re-apply the segment-specific extraction prompt to the redetected region; this strategy may be preferred for missing-value failures attributable to incorrect boundary detection. (iii) code/regex fallback—for data items whose values follow deterministic patterns (numeric ranges, date formats, coded identifiers), the refinement service may bypass the LLM entirely and apply a hand-crafted regex or parse rule to extract the value directly from the raw segment text; this strategy may be preferred for wrong-format and out-of-range failures where the LLM introduced unnecessary variation. (iv) segment decomposition—for complex segments that contain multiple overlapping field values, the refinement service may decompose the segment into smaller, non-overlapping sub-regions, apply independent extraction prompts to each sub-region, and merge the resulting field values; this strategy may be preferred for structural inconsistency failures where a single monolithic extraction prompt proved insufficient.

After each re-extraction attempt, the refinement service may merge the updated data values into the extracted data of the segment and re-execute the originating guard rule against the updated data values to obtain a revised confidence score. If the revised score meets or exceeds the acceptance threshold, the refinement service may issue a PASS result and may output the updated data values. If, however, the revised confidence score remains below threshold, the refinement service checks whether the maximum refinement iteration count for the current phase has been reached. If iterations remain, the refinement service may advance to the next strategy in the configured sequence and execute another re-extraction attempt. If the maximum iteration count is exhausted without achieving the acceptance threshold, the affected fields may be annotated with a LOW CONFIDENCE flag, a human-review notification may be generated identifying the specific guard rule that failed, and failure reason, and the segment including the failed data. A refinement service may include a human-review notification annotation in the final extraction output, ensuring that downstream consumers and human reviewers are informed about which fields require attention and why.

For every field processed by the refinement service, a re-extraction audit record may be appended to the segment extracted data. Each record may include: the originating guard rule identifier and type; the failure mode classification; the sequence of strategies attempted, in order; the input data value and output data value for each attempt; the revised confidence score produced by the re-evaluation guard rule run after each attempt; and the final disposition (PASS, ESCALATE, or MAX ITERATIONS). This audit trail may be incorporated into the final output report alongside the post-processing report and final extracted data, providing a complete, reproducible account of every re-extraction decision for regulatory traceability and continuous improvement analysis.

User feedback module 230 may detect and capture real-time user feedback regarding the accuracy and completeness of the extracted data. The user feedback may include corrections, annotations, approval, rejection, and confidence override made by an authorized reviewer on the extracted data. User feedback module 230 may normalize various forms of feedback, including manual corrections, flags, and comments, into a standardized format. User feedback module 230 may transform the user feedback into a normalized, semantically indexed knowledge base stored in a vector database 250. Vector database 250 may be a searchable repository of historical corrections and expert preferences that may serve as a contextual knowledge base for an onboarding module 210.

User feedback module 230 may monitor four categories of user feedback: manual data corrections, in which a reviewer directly edits the value of an extracted data; annotations and comments, in which a reviewer attaches free-text explanatory notes to a data item or segment; approval and rejection actions, in which a reviewer accepts or rejects all extracted data of a clinical report; and confidence overrides, in which a reviewer manually adjusts a confidence score for a data item. For each feedback event, user feedback module 230 may record a raw event payload including: an event type; a session identifier; a template identifier; a segment type and data field name; the original extracted value; the user-provided value or action; and a timestamp.

User feedback module 230 may normalize raw event payload by resolving the template identifier, segment type, and data field name are resolved to their canonical forms and map the event type to a standard feedback type. The standard feedback type types may include:
VALUE_CORRECTION—the reviewer replaced an incorrect extracted data value with the correct one.
FORMAT_CORRECTION—the reviewer corrected the format or representation of a data value without changing its substance.
SEMANTIC FLAG—the reviewer indicated that a data value was semantically incorrect without providing a replacement.
APPROVAL—the reviewer confirmed that the extracted data value was correct.
REJECTION—the reviewer rejected the entire record.

Normalization may further include generating a feedback record including all canonical fields-template identifier, segment type, data field name, original extracted value, corrected value, feedback type, reviewer identifier, and timestamp, together with two derived fields where applicable: the confidence delta, which may be the difference between the original system confidence score and the reviewer-assigned confidence.

Once a feedback record has been normalized, user feedback module 230 may generate a dense vector embedding representing the semantic content of the normalized feedback record. The embedding is computed by a domain-tuned sentence-transformer model that receives as input a serialized representation of the feedback record that encodes the segment type, field name, original value, corrected value, and feedback type as a structured natural-language string. The resulting fixed-dimension embedding vector (typically 768 dimensions) captures the semantic relationship between the original extraction error and the correct value, positioning similar types of correction close together in the embedding space regardless of superficial textual differences. The embedding vector and its associated feedback record metadata may be written to vector database 250 as a single indexed entry. In some embodiments, vector database 250 may maintain a hierarchical navigable small-world (HNSW) approximate nearest-neighbor index partitioned by template identifier and data field name, enabling highly efficient scoped similarity searches that are constrained to the relevant template and data field without scanning the entire feedback corpus. Vector database 250 may support versioned snapshots for audit purposes, soft-delete for feedback retraction, and upsert (update+insert) semantics for correction amendments.

A similarity search API is exposed by user feedback module 230 to other pipeline components. A caller may submit a query consisting of: a query vector, which is an embedding of the current extraction context (computed using the same sentence-transformer model applied to a serialized representation of the new extraction input); an optional metadata filter specifying the template identifier, data field name, and feedback type; and a top-K (K>=1) parameter specifying the maximum number of similar feedback records to return. The API may execute an approximate nearest-neighbor search against the HNSW index, apply the metadata filter as a pre-filter or post-filter depending on selectivity, and return the top-K most similar feedback records ranked by a similarity score such as the cosine similarity score. A configurable similarity threshold, set per template in the extraction configuration, ensures that only sufficiently similar historical corrections are returned; results below the threshold are suppressed to prevent the injection of irrelevant correction context into downstream consumers.

The top-K most similar feedback records may be clustered to clusters that share a common underlying correction pattern. Clustering may be performed, for example, using a density-based algorithm (such as hierarchical density-based spatial clustering of applications with noise (HDBSCAN)) applied to the embedding vectors of the retrieved feedback records, which groups feedback records that are semantically proximate in the embedding space without requiring a pre-specified number of clusters. Each resulting cluster may be characterized by a cluster signature including the representative template identifier, data field name, dominant feedback type, and a correction delta summary that describes the nature of the transformation shared across cluster members. Clusters that do not meet the configured minimum frequency threshold, e.g., the minimum number of independent correction events required before a correction pattern is considered actionable may be discarded, preventing the learning service from acting on idiosyncratic or erroneous one-off corrections. Clusters that pass the frequency threshold may be forwarded to the appropriate learning path, e.g., post-processing rule synthesis and/or extraction prompt optimization, based on the dominant feedback type in the cluster signature.

Post-processing rule synthesis may operate on clusters whose dominant feedback type is VALUE_CORRECTION or FORMAT_CORRECTION. Those clusters may be forwarded to post processing module 228 for transformation rule synthesis. Post processing module 228 may examine the cluster signature and a sample of individual correction records to identify the underlying transformation type. Post processing module 228 may classify the transformation into one of four categories: format transformation (e.g. date or numeric format standardization), unit conversion (e.g. normalization of measurement units to a canonical representation), controlled vocabulary mapping (e.g. replacement of gene symbol aliases with canonical identifiers), or type coercion (e.g. conversion of free-text numeric strings to typed values). The correction pattern, together with a set of representative correction examples may be submitted to the LLM rule synthesizer. An LLM rule synthesizer may construct a structured prompt presenting the correction examples and the identified transformation type, and instruct an LLM of one or more LLMs 240 to generate a candidate post-processing rule (e.g., a Python rule) that captures the observed transformation as an executable function, accompanied by a natural-language explanation and indication of the relevant data filed to which this rule should be applied. Candidate transformation rules may be reviewed by a human operator. Approved transformation rules may be included in the pipeline instructions and become immediately active for future extraction runs against the relevant clinical report format.

Extraction prompt optimization may operate on all clusters, regardless of the feedback type. Extraction prompt optimization may contribute to extraction prompt optimization through selecting few-shot examples for inclusion in the extraction prompts generated by onboarding module 210. Extraction prompt optimization may identify the highest-quality correction examples from the feedback record corpus by ranking candidates on three criteria: semantic similarity score (proximity to the current extraction context in embedding space), recency (more recent corrections are preferred, reflecting the most current reviewer preferences), and reviewer authority weight (corrections from domain experts or lead reviewers are up-weighted relative to junior reviewer corrections). The selected examples are deduplicated and trimmed to fit within the LLM extraction prompt, and formatted as structured input-output pairs that can be directly injected into the LLM extraction prompt as few-shot demonstrations. Onboarding module 210 may maintain versioned extraction prompt variants per clinical report format, conducts A/B testing of prompt variants on labeled clinical reports to measure quality improvements, and automatically promotes the best-performing extraction prompt variant as the active extraction prompt for the clinical report format.

Vector database 250 and similarity search API provide self-learning capabilities to both the onboarding module 210 and the extraction module 220. Onboarding module 210 may query the similarity search API with embeddings derived from batches of recent extraction inputs to retrieve clusters of similar past corrections. Onboarding module 210 may use these clusters as evidence to identify recurring correction patterns and synthesize new transformation rules via the LLM rule synthesizer (included in onboarding module 210). Extraction module 220 may query the API at extraction time, before applying extraction prompts to retrieve the top-K most similar historical corrections for the current segment and data field. The retrieved corrections may be injected as few-shot examples into the segment extraction prompt, which may incorporate accumulated reviewer expertise into the extraction process without modifying model weights.

Using the similarity search API by onboarding module 210 and by extraction module 220 as described may improve the technology of data extraction by generating extraction prompts and transformation rules that are refined using a closed self-improvement loop that incorporates user feedback into transformation rules and extraction prompts.

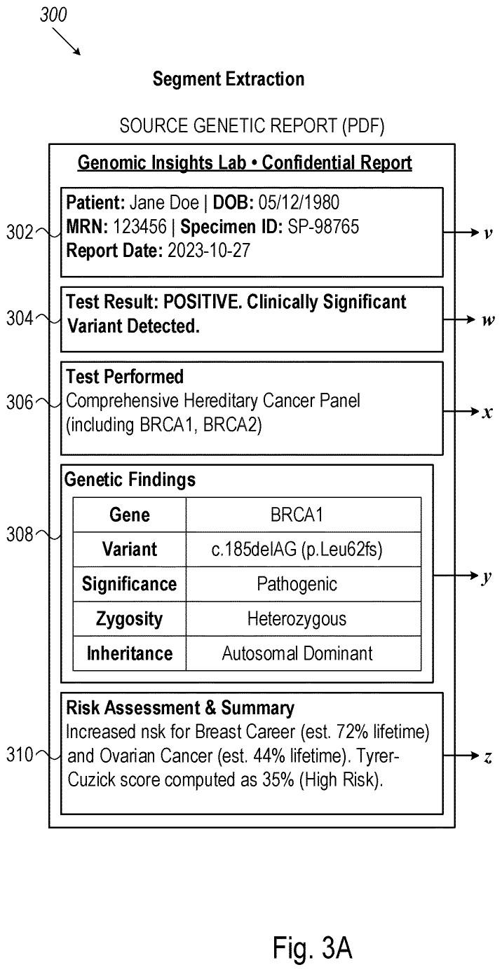

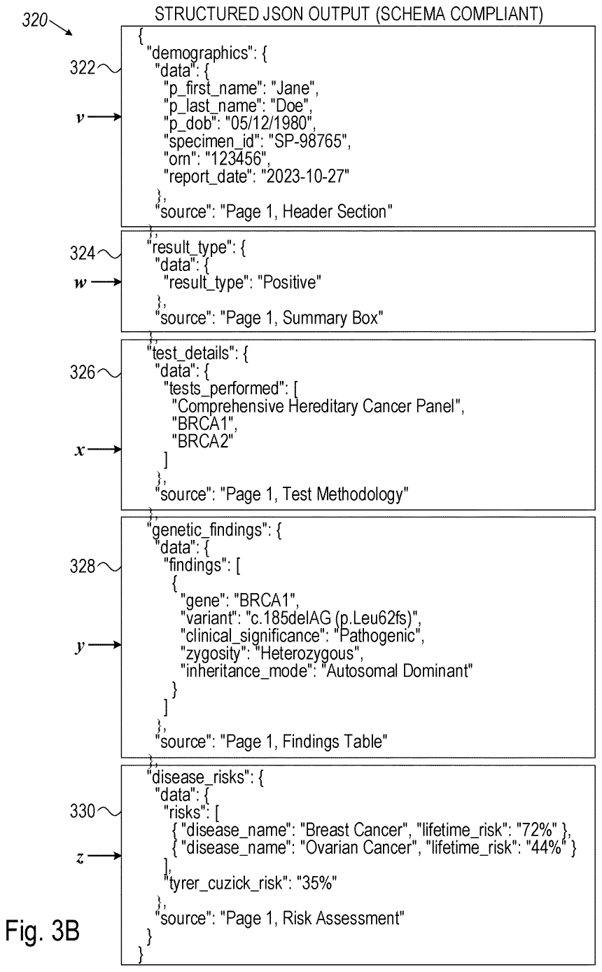

Reference is now made to FIGS. 3A-3B, which provide an example of a clinical report 300 and extracted data 304, according to embodiments of the invention. In the example of FIGS. 3A-3B, clinical report 300 is segmented into segments 302-310 and the extracted data 320 includes data extracted from each segment 322-330.

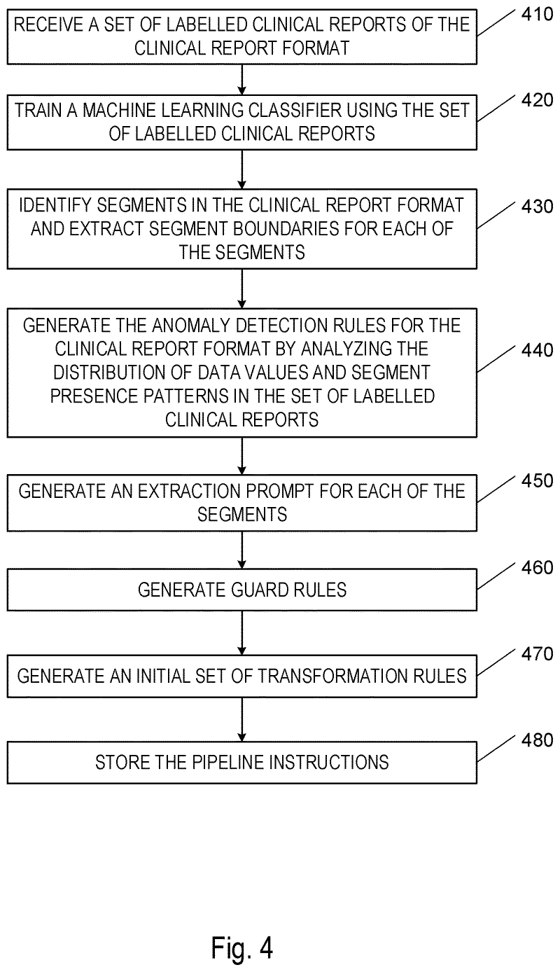

Reference is now made to FIG. 4, which is a flowchart of a method for preparing pipeline instructions for a new clinical report format, according to embodiments of the invention. While in some embodiments the operations of FIG. 4 are carried out using systems as shown in FIGS. 1 and 2, in other embodiments other systems and equipment can be used.

In operation 410, a processor (e.g., processor 105 depicted in FIG. 1 executing code to carry out embodiments of the method for extracting medical data from clinical reports) may receive a set of labelled clinical reports of the clinical report format, where the label may include an output schema that defines the structure of the clinical report, including true segment boundaries and data field names, and the true data values.

In operation 420, the processor may train an ML classifier using the set of labelled clinical reports. The processor may train an ML classifier by performing a structural analysis of the set of labelled clinical reports to generate a template structure model, extracting candidate features from the template structure model, and training the machine learning classifier using the candidate features to determine whether an examined clinical report conforms to the clinical report format. The candidate features may include header and footer text patterns, logo position and dimensions, characteristic font families, recurring table structures, and clinical report statistics. The ML classifier may be one of a support vector machine or a random forest classifier, an ML or NLP algorithms trained on labeled clinical reports, or an LLM based classifier with few-shot prompting, executed by, for example, AWS comprehend (an AWS NLP service). The classifier configuration may include feature weights, the decision boundary, and a calibrated confidence threshold below which a classification result is considered ambiguous.

In operation 430, the processor may identify segments in the clinical report format and extracting segment boundaries for each of the segments. The processor may extract the segment boundaries by analyzing the set of labelled clinical reports to extract textual and positional signals that delimit segment types within the clinical report format, generating a set of regular expression boundary rules and an LLM segment detection prompt that instructs the LLM to identify segment start and end markers, validating the set of regular expression boundary rules and the LLM segment detection prompt against a second set of labelled clinical reports having the clinical report format, and iteratively adjusting the set of regular expression boundary rules and the LLM segment detection prompt until a minimum coverage threshold is achieved.

In operation 440, the processor may generate anomaly detection rules for the clinical report format by analyzing the distribution of data values and segment presence patterns in the set of labelled clinical reports. The processor may generate the anomaly detection rules by identifying which segments and data fields are mandatory in the clinical report format, generating a presence checklist that defines which absences constitute anomalies, deriving plausible value ranges and canonical format patterns for data items in the clinical report format from the set of labelled clinical reports, and generating a structured anomaly detection configuration comprising missing-segment alert rules based on the presence checklist, out-of-range value flags based on the plausible value ranges, and format violation rules based on the canonical format patterns.

In operation 450, the processor may generate, from the set of labelled clinical reports using an LLM, an extraction prompt for each of the segments, where each of the extraction prompts is configured to extract data from a segment of the clinical report format. An extraction prompt may include a data field schema and an expected output format. The processor may generate each of the extraction prompts by iteratively refining the extraction prompt by applying the extraction prompt to the associated segment to extract data from the segment, evaluating the extracted data item against the true data values, and using an LLM to refine the prompt based on the evaluation.

In operation 460, the processor may analyze the distribution of the true data values to generate guard rules that enable define value ranges for extracted data. The generated guard rules may include an LLM guard prompt that instructs the LLM to evaluate semantic plausibility, a code guard function that performs deterministic range and type checking, and a regular expression guard pattern that validates format compliance. Other guard rules may be generated or added manually. The guard rules may include segment guard rules and format guard rules.

In operation 470, the processor may generate an initial set of transformation rules based on data patterns observed in the set of labelled clinical reports. The initial set of transformation rules may include rules for formatting conventions, transformation rules for unit normalisation, transformation rules for date format standardisation, transformation rules for vocabulary mappings, and type coercion transformation rules. Other transformation rules may be generated or added manually.

In operation 480, the processor may compose, from the final set of regular expression boundary rules and LLM segment detection prompt, the generated extraction prompts, the guard rules, the transformation rules, and other relevant metadata such as the output scheme, pipeline instructions that are associated with the clinical report format, and store the pipeline instructions in a pipeline instructions repository.

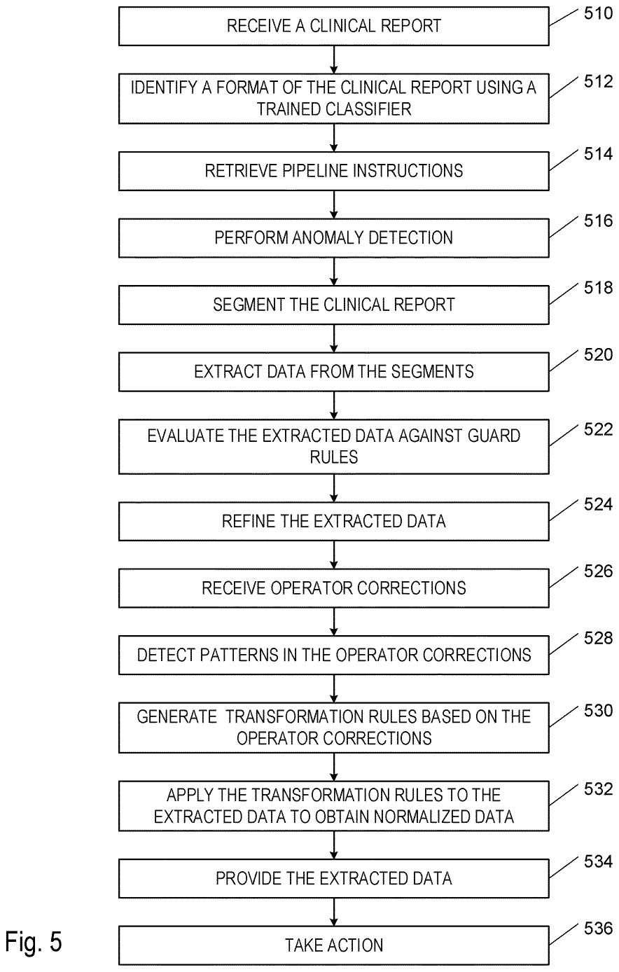

Reference is now made to FIG. 5, which is a flowchart of a method for extracting medical data from clinical reports, according to embodiments of the invention. While in some embodiments the operations of FIG. 5 are carried out using systems as shown in FIGS. 1 and 2, in other embodiments other systems and equipment can be used.

In operation 510, a processor (e.g., processor 105 depicted in FIG. 1 executing code to carry out embodiments of the method for extracting medical data from clinical reports) may receiving a clinical report for analysis and data retrieval. The report may be in any applicable format, e.g., PDF, DODX Word documents, electronic publication (EPUB) formats, HyperText markup language (HTML) pages, image-based formats, flipbook formats, etc.

In operation 512, the processor may identify a format of the clinical report using a trained classifier, e.g., the classifier trained in operation 420. In operation 514, the processor may retrieve pipeline instructions associated with the report format, e.g., the processor may retrieve the pipeline instructions generated in operation 480 from the pipeline instruction repository. The processor may use data in the pipeline instructions for performing operations 516-530. In operation 516, the processor may perform anomaly detection to detect whether there are deviations from the report template within the clinical report using anomaly detection rules. The processor may provide an alert in case deviations are detected.

In operation 518, the processor may segment the clinical report into segments using the report template included in the pipeline instructions. In operation 520, the processor may extract data from each segment by applying at least one extraction prompt from the pipeline instructions to at least one LLM (e.g., from one or more LLM 240), to generate raw extracted data. In operation 522, the processor may evaluate the extracted data against guard rules, and in operation 524 the processor may refine the extracted data based on the guard rule evaluation. The guard rules may include segment guard rules and template guard rules, presence guards that verify that all data fields marked as mandatory in the pipeline instructions are populated with non-null values in the extracted data, range guard rules that verify that numeric data in the extracted data fall within clinically plausible ranges defined in the pipeline instructions, and format guards that verify that string data fields in the extracted data conform to expected patterns. The types of guard rules may include:
LLM-based guard rules that include an evaluation prompt comprising the extracted data and the segment from which the extracted data were extracted as context,
code-based guard rules that apply logic rules to the extracted data,
regex-based guard rules that validate format of extracted string data fields within the data by matching the extracted string data fields against one or more regular expression patterns,
rule-based guard rules that evaluate the extracted data against domain knowledge bases;
generic guard rules that validate date of birth (DOB), patient name and general results, and
clinical indication-specific guard rules that validate variant names and gene names.

The processor may refine a certain extracted data item in case evaluating the data item against its associated guard rule fails. Refining may include providing to the at least one LLM (e.g., at least one LLMs 240) the first extracted data item, the first guard rule, the segment including the first extracted data item and the extraction prompt applied to the at least one LLM to extract the first extracted data item, updating, by the at least one LLM, the extraction prompt applied to the at least one LLM to extract the first extracted data item, and applying the updated extraction prompt to the segment including the extracted data item to obtain a refined extracted data item.

Refining the extracted data may include:
for cases of semantic mismatch and missing value failure modes: updating the extraction prompt used to extract the extracted data item to comprise a guard failure reason and constrain the extracted data to the expected output format, and applying the updated extraction prompt to the segment including the first extracted data item to extract a refined first data item from the updated segment,
for missing-value failures: repeating segmenting the clinical report into segments with adjusted boundary parameters, and, for the updated segment including the first extracted data item, repeating applying the extraction prompt to the at least one LLM to extract a refined first data item from the updated segment;
for data that follow deterministic patterns-applying regex rules to the segment including the first extracted data item to extract a refined first data item from the updated segment,
for complex segments that contain multiple overlapping data: segmenting the segment including the first extracted data item into smaller, non-overlapping sub-segments, applying independent extraction prompts to each sub-segment, and merging the resulting data to extract a refined first data item from the updated segment.

In operation 526, the processor may receive operator corrections made to previously extracted data. In operation 528, the processor may detect, using one or more LLMs, patterns in the operator corrections. In operation 530, the processor may generate, using the at least one LLMs, transformation rules that encode the patterns in the operator corrections into transformation rules, and in operation 532, the processor may apply the transformation rules to the extracted data to obtain normalized data. The processor may apply transformation rules that target non-overlapping extracted data in parallel and apply sequentially transformation rules that target the same extracted data. The types of transformation rule may include:
code transformation rules that encode transformation logic as executable software code,
regular expressions (regex) transformation rules that apply regular expression patterns to the data,
prompt transformation rules that comprise an LLM prompt that describes the transformation rule,
LLM-generated rules that are produced by an LLM rule synthesizer and encode transformation patterns inferred from operator correction behavior.

In some embodiments, the processor may evaluate the normalized data against a second set of guard rules and refine the normalized data based on the evaluation of the normalized data against a second set of guard rules. Refining the normalized data based on the evaluation of the normalized data against the second set of guard rules may include, in case evaluating a first normalized data item against a second guard rule fails, providing the first normalized data item, the second guard rule, the extracted data item before normalization, and the transformation rule that was applied to the extracted data item to perform the normalization to the at least one LLM, updating, by the at least one LLM, the transformation rule that was applied to the extracted data item to perform the normalization; and applying the updated transformation rule to the extracted data item before normalization to obtain a refined normalized data item.

In operation 534, the processor may provide the final output report including the final extracted data and refinement and transformation history to a user or to other computerized systems such as clinical and analytical systems, or store the final extracted data in a clinical report database.

In operation 536, an action may be taken based on or using the final extracted data. For example, the final extracted data and post-processing outputs may be used to calculate a patient's genetic risk (e.g., during or prior to pregnancy) and generate, based on this analysis, a clinical report that includes the final extracted data, e.g., genetic findings and their clinical interpretation. The clinical report may be sent automatically, for example, in the form of a push notification, to a healthcare provider for review. Similarly, the final extracted data may be sent to the patient. In case the results indicate certain medical conditions, an alert may be generated and automatically sent to the healthcare provider, e.g., in the form of a push notification. Embodiments may recommend follow-up actions, such as scheduling a face-to-face consultation, performing additional diagnostic tests or providing a referral to a specialist. Action may include controlling data flow, e.g., restricting or enabling sharing of the final extracted data based on risk level associated with the final extracted data, based on specific findings in the final extracted data, or based on the validation status. For example, embodiments may automatically route or send the final extracted data that includes specific values or findings to authorized operators and/or restrict access to the final extracted data form other operators that are unauthorized to receive final extracted data that includes those specific values.

In some embodiments, a message may be generated automatically containing the extracted data and possibly medical interpretation, and the message may be transmitted to relevant users, e.g., care givers, over a computer network in real time, so that each user has immediate access to up-to-date patient information.

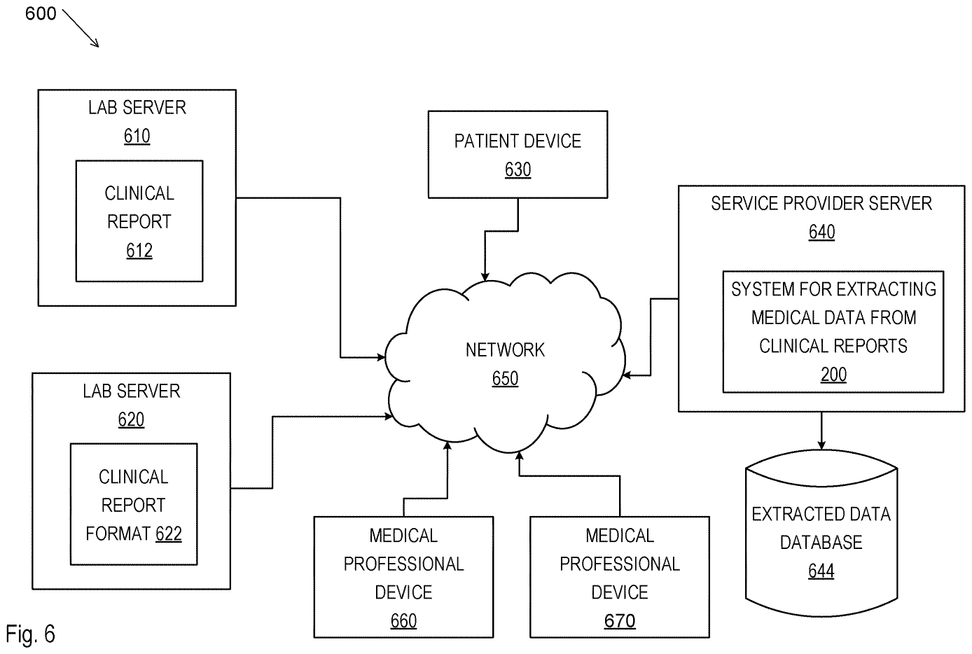

Reference is made to FIG. 6, which is a computerized medical system 600 according to some embodiments. According to some embodiments, laboratory (LAB) server 610, LAB server 620, service provider server 640, patient device 630, medical professional device 660 and medical professional device 670 may include components of computing device 100. Extracted data database 644 may be a storage device (e.g., storage 130 depicted in FIG. 1) used for storing the final extracted data, as required by the application. While presented with a direct connection to service provider server 640, extracted data database 644 may be connected to service provider server 640 via network 650. It is noted that system 600 is presented as an example only, and that computerized medical systems may have other architectures and include a plurality of patient devices 630, LAB servers 610, 620, service provider servers 640 medical professional devices 660 and 670.

Network 650 may include any type of network or combination of networks available for supporting communication between laboratory (LAB) server 610, LAB server 620, service provider server 640, patient device 630, medical professional device 660 and medical professional device 670. Networks 650 may include for example, a wired, wireless, fiber optic, or any other type of connection, a local area network (LAN), a virtual private network (VPN), a wide area network (WAN), the Internet and intranet networks, etc.

LAB server 610, LAB server 620 may produce clinical reports 612 and 622 for a patient, e.g., the patient associated with patient device 630. Clinical report 612 may be in a first format, different from the format of clinical report 622. Both clinical reports 612 and 622 may be sent to service provider server 640, e.g., over network 650 or by other means. Clinical reports 612 and 622 may be analyzed by system 200 for extracting medical data from clinical reports, operated by service provider server 640 to extract medical data from clinical reports 612 and 622, as disclosed herein. Once the data is extracted and a final output report is generated, the final output report may be generated and the final extracted data may be stored in extracted data database 644, in any applicable format. Thus, while each of clinical reports 612 and 622 may have a different format, system 200 may extract the data from clinical reports 612 and 622 and store the final extracted data in a unified format that is accessible to service provider server 640. In addition, service provider server 640 may distribute the final extracted data among patient device 630, medical professional device 660 and medical professional device 670, according to policies defined by service provider server 640 for the different extracted data types.

For example, service provider server 640 may use the final extracted data and other data included in the final output report to generate a clinical report that includes the final extracted data, e.g., genetic findings and their clinical interpretation. Service provider server 640 may send the clinical report automatically, for example, in the form of a push notification, to patient device 630, medical professional device 660 and/or medical professional device 670. In case the results indicate certain medical conditions, service provider server 640 may generate an alert and automatically send the alert to medical professional device 660 and/or medical professional device 670, e.g., in the form of a push notification. Service provider server 640 may recommend follow-up actions, such as scheduling a face-to-face consultation, performing additional diagnostic tests or providing a referral to a specialist. Service provider server 640 may control data flow, e.g., restrict or enable sharing of the final extracted data patient device 630, medical professional device 660 and/or medical professional device 670 based on risk level associated with the final extracted data, based on specific findings in the final extracted data, or based on the validation status. For example, service provider server 640 may automatically route or send the final extracted data that includes specific values or findings to authorized operators, e.g., medical professional device 660 and/or restrict access to the final extracted data form other operators, e.g., medical professional device 670, that are unauthorized to receive extracted data that includes those specific values.

In some embodiments, service provider server 640 may automatically generate a message containing the final extracted data and possibly other data from the final output report and/or medical interpretation, and transmit the message to relevant users, e.g., devices of care givers such as patient device 630, medical professional device 660 and/or medical professional device 670, over computer network 650 in real time, so that each user has immediate access to up-to-date patient information.

One skilled in the art will realize the embodiments may be embodied in other specific forms without departing from the spirit or essential characteristics thereof. The foregoing embodiments are therefore to be considered in all respects illustrative rather than limiting of the embodiments described herein. Scope of the embodiments is thus indicated by the appended claims, rather than by the foregoing description, and all changes that come within the meaning and range of equivalency of the claims are therefore intended to be embraced therein.

In the foregoing detailed description, numerous specific details are set forth in order to provide an understanding of the embodiments. However, it will be understood by those skilled in the art that the embodiments can be practiced without these specific details. In other instances, well-known methods, procedures, and components, modules, units and/or circuits have not been described in detail so as not to obscure the embodiments. Some features or elements described with respect to one embodiment can be combined with features or elements described with respect to other embodiments.

Although embodiments of the embodiments are not limited in this regard, discussions utilizing terms such as, for example, “processing,” “computing,” “calculating,” “determining,” “establishing”, “analyzing”, “checking”, or the like, can refer to operation(s) and/or process(es) of a computer, a computing platform, a computing system, or other electronic computing device, that manipulates and/or transforms data represented as physical (e.g., electronic) quantities within the computer's registers and/or memories into other data similarly represented as physical quantities within the computer's registers and/or memories or other information non-transitory storage medium that can store instructions to perform operations and/or processes.

Although embodiments of the embodiments are not limited in this regard, the terms “plurality” and “a plurality” as used herein can include, for example, “multiple” or “two or more”. The terms “plurality” or “a plurality” can be used throughout the specification to describe two or more components, devices, elements, units, parameters, or the like. The term set when used herein can include one or more items. Unless explicitly stated, the method embodiments described herein are not constrained to a particular order or sequence. Additionally, some of the described method embodiments or elements thereof can occur or be performed simultaneously, at the same point in time, or concurrently.

1. A computer method for extracting medical data from clinical reports, the method comprising, using a processor:
identifying a format of the clinical report using a trained classifier;
retrieving pipeline instructions associated with the report format, wherein the pipeline instructions comprise a report template, at least one prompt and guard rules, and using the pipeline instruction:
segmenting the clinical report into segments using the report template;
for each segment, extracting data from the segment by applying the at least one extraction prompt to at least one large language model (LLM);
evaluating the extracted data against guard rules;
refining the extracted data based on the guard rule evaluation;
receiving operator corrections made to previously extracted data;
detecting, using the at least one LLM, patterns in the operator corrections;
generating, using the at least one LLM, transformation rules that encode the patterns in the operator corrections into transformation rules; and
applying the transformation rules to the extracted data to obtain normalized data.

2. The method of claim 1, wherein the guard rules are selected from: presence guards that verify that all data fields marked as mandatory in the pipeline instructions are populated with non-null values in the extracted data, range guard rules that verify that numeric data in the extracted data fall within clinically plausible ranges defined in the pipeline instructions, and format guards that verify that string data fields in the extracted data conform to expected patterns.

3. The method of claim 1, wherein types of the guard rules are selected from:
LLM-based guard rules that comprise an evaluation prompt comprising the extracted data and the segment from which the extracted data were extracted as context,
code-based guard rules that apply logic rules to the extracted data,
regex-based guard rules that validate format of extracted string data fields within the data by matching the extracted string data fields against one or more regular expression patterns,
rule-based guard rules that evaluate the extracted data against domain knowledge bases;
generic guard rules that validate date of birth (DOB), patient name and general results, and
clinical indication-specific guard rules that validate variant names and gene names.

4. The method of claim 1, wherein the transformation rules that target non-overlapping extracted data are applied in parallel and the transformation rules that target the same extracted data are applied sequentially.

5. The method of claim 1, wherein types of the transformation rules are selected from:
code transformation rules that encode transformation logic as executable software code,
regular expressions (regex) transformation rules that apply regular expression patterns to the data,
prompt transformation rules that comprise an LLM prompt that describes the transformation rule,
LLM-generated rules that are produced by an LLM rule synthesizer and encode transformation patterns inferred from operator correction behavior.

6. The method of claim 1, wherein refining the extracted data comprises, in case of evaluating a first extracted data item against a first guard rule fails:
providing to the at least one LLM the first extracted data item, the first guard rule, the segment including the first extracted data item and the extraction prompt that was applied to the at least one LLM to extract the first extracted data;
updating, by the at least one LLM, the extraction prompt applied to the at least one LLM to extract the first extracted data item; and
applying the updated extraction prompt to the segment including the extracted data item to obtain a refined extracted data item.

7. The method of claim 1, wherein refining the extracted data based on the evaluation, in case evaluating a first extracted data item against a first guard rule fails, comprises any one of:
for cases of semantic mismatch and missing value failure modes: updating the extraction prompt used to extract the extracted data item to comprise a guard failure reason, constraining the extracted data to the expected output format, and applying the updated extraction prompt to the segment including the first extracted data item to extract a refined first data item from the updated segment,
for missing-value failures: repeating segmenting the clinical report into segments with adjusted boundary parameters, and, for the updated segment including the first extracted data item, repeating applying the extraction prompt to the at least one LLM to extract a refined first data item from the updated segment;
for data that follow deterministic patterns-applying regex rules to the segment including the first extracted data item to extract a refined first data item from the updated segment,
for complex segments that contain multiple overlapping data items: segmenting the segment including the first extracted data item into smaller, non-overlapping sub-segments, applying independent extraction prompts to each sub-segment, and merging the resulting data to extract a refined first data item from the updated segment.

8. The method of claim 1, comprising:
evaluating the normalized data against a second set of guard rules; and
refining the normalized data based on the evaluation of the normalized data against a second set of guard rules.

9. The method of claim 8, wherein refining the normalized data based on the evaluation of the normalized data against the second set of guard rules comprises, in case evaluating a first normalized data item against a second guard rule fails:
providing the first normalized data item, the second guard rule, the extracted data item before normalization, and the transformation rule that was applied to the extracted data item to perform the normalization to the at least one LLM;
updating, by the at least one LLM, the transformation rule that was applied to the extracted data item to perform the normalization; and
applying the updated transformation rule to the extracted data item before normalization to obtain a refined normalized data item.

10. The method of claim 1, comprising:
detecting whether there are deviations from the report template within the clinical report using anomaly detection rules, before segmenting the clinical report; and
providing an alert in case deviations are detected.

11. The method of claim 1, comprising, in a preparation stage:
receiving a set of labelled clinical reports of the clinical report format, wherein the label comprising true segment boundaries and true data values; and
generating, using at least one machine learning model, the pipeline instructions for the clinical report format.

12. The method of claim 1, wherein generating the pipeline instructions for the clinical report format comprises:
training a machine learning classifier using the set of labelled clinical reports;
identifying segments in the clinical report format and extracting segment boundaries for each of the segments;
generating anomaly detection rules for the clinical report format;
generating, from the set of labelled clinical reports using a large language model (LLM), the at least one extraction prompt for each of the segments, wherein each of the extraction prompts is configured to extract data from a segment of the clinical report format;
analysing the distribution of the true data values to generate guard rules that enable define value ranges for extracted data; and
generating an initial set of transformation rules based on data patterns in the set of labelled clinical reports.

13. A device comprising:
a memory; and
one or more processors configured to:
identify a format of the clinical report using a trained classifier;
retrieve pipeline instructions associated with the report format, wherein the pipeline instructions comprise a report template, at least one prompt and guard rules, and using the pipeline instruction:
segment the clinical report into segments using the report template;
for each segment, extract data from the segment by applying the at least one extraction prompt to at least one large language model (LLM);
evaluate the extracted data against guard rules;
refine the extracted data based on the guard rule evaluation;
receive operator corrections made to previously extracted data;
detect, using the at least one LLM, patterns in the operator corrections;
generate, using the at least one LLM, transformation rules that encode the patterns in the operator corrections into transformation rules; and
apply the transformation rules to the extracted data to obtain normalized data.

14. The device of claim 13, wherein types of the guard rules are selected from:
LLM-based guard rules that comprise an evaluation prompt comprising the extracted data and the segment from which the extracted data were extracted as context,
code-based guard rules that apply logic rules to the extracted data,
regex-based guard rules that validate format of extracted string data fields within the data by matching the extracted string data fields against one or more regular expression patterns,
rule-based guard rules that evaluate the extracted data against domain knowledge bases;
generic guard rules that validate date of birth (DOB), patient name and general results, and
clinical indication-specific guard rules that validate variant names and gene names.

15. The device of claim 13, wherein types of the transformation rules are selected from:
code transformation rules that encode transformation logic as executable software code,
regular expressions (regex) transformation rules that apply regular expression patterns to the data,
prompt transformation rules that comprise an LLM prompt that describes the transformation rule,
LLM-generated rules that are produced by an LLM rule synthesizer and encode transformation patterns inferred from operator correction behavior.

16. The device of claim 13, wherein the one or more processors are configured to refine the extracted data by, in case of evaluating a first extracted data item against a first guard rule fails:
providing to the at least one LLM the first extracted data item, the first guard rule, the segment including the first extracted data item and the extraction prompt that was applied to the at least one LLM to extract the first extracted data;
updating, by the at least one LLM, the extraction prompt applied to the at least one LLM to extract the first extracted data item; and
applying the updated extraction prompt to the segment including the extracted data item to obtain a refined extracted data item.

17. The device of claim 13, wherein the one or more processors are configured to refine the extracted data based on the evaluation, in case evaluating a first extracted data item against a first guard rule fails, by any one of:
for cases of semantic mismatch and missing value failure modes: updating the extraction prompt used to extract the extracted data item to comprise a guard failure reason, constraining the extracted data to the expected output format, and applying the updated extraction prompt to the segment including the first extracted data item to extract a refined first data item from the updated segment;
for missing-value failures: repeating segmenting the clinical report into segments with adjusted boundary parameters, and, for the updated segment including the first extracted data item, repeating applying the extraction prompt to the at least one LLM to extract a refined first data item from the updated segment; and
for data that follow deterministic patterns-applying regex rules to the segment including the first extracted data item to extract a refined first data item from the updated segment,
for complex segments that contain multiple overlapping data items: segmenting the segment including the first extracted data item into smaller, non-overlapping sub-segments, applying independent extraction prompts to each sub-segment, and merging the resulting data to extract a refined first data item from the updated segment.

18. The device of claim 13, wherein the one or more processors are configured to:
evaluate the normalized data against a second set of guard rules; and
refine the normalized data based on the evaluation of the normalized data against a second set of guard rules.

19. The device of claim 18, wherein the one or more processors are configured to refine the normalized data based on the evaluation of the normalized data against the second set of guard rules by, in case evaluating a first normalized data item against a second guard rule fails:
providing the first normalized data item, the second guard rule, the extracted data item before normalization, and the transformation rule that was applied to the extracted data item to perform the normalization to the at least one LLM;
updating, by the at least one LLM, the transformation rule that was applied to the extracted data item to perform the normalization; and
applying the updated transformation rule to the extracted data item before normalization to obtain a refined normalized data item.

20. The device of claim 13, wherein the one or more processors are configured to, in a preparation stage:
receive a set of labelled clinical reports of the clinical report format, wherein the label comprising true segment boundaries and true data values; and
generate, using at least one machine learning model, the pipeline instructions for the clinical report format.
